(19) United States
(12) Patent Application Publication
(10) Pub. No.: US 2025/0034545 A1
(43) Pub. Date: Jan. 30, 2025

(54) MODIFICATIONS OF PEPTIDE COMPOSITIONS TO INCREASE STABILITY AND DELIVERY EFFICIENCY

(71) Applicant: KAI PHARMACEUTICALS, INC., South San Francisco, CA (US)

(72) Inventor: Derek Maclean, Los Altos, CA (US)

(21) Appl. No.: 18/590,842

(22) Filed: Feb. 28, 2024

Related U.S. Application Data
(63) Continuation of application No. 17/749,004, filed on May 19, 2022, now abandoned.
Application No. 17/749,004 is a continuation of application No. 16/933,749, filed on Jul. 20, 2020, now abandoned.
Application No. 16/933,749 is a continuation of application No. 16/120,156, filed on Aug. 31, 2018, now abandoned.
Application No. 16/120,156 is a division of application No. 14/991,823, filed on Jan. 8, 2016, now Pat. No. 10,077,438.
Application No. 14/991,823 is a division of application No. 13/305,685, filed on Nov. 28, 2011, now Pat. No. 9,255,124.
Application No. 13/305,685 is a division of application No. 12/017,985, filed on Jan. 22, 2008, now Pat. No. 8,067,532.
(60) Provisional application No. 60/945,285, filed on Jun. 20, 2007.
(60) Provisional application No. 60/881,419, filed on Jan. 19, 2007.

(51) Int. Cl. C12N 9/96; A61K 38/10; A61K 47/64; C07K 7/00; C12N 9/12

(52) U.S. Cl. CPC C12N 9/96; A61K 38/10; A61K 47/64; A61K 47/645; C07K 7/00; C12N 9/12; C12Y 207/11013

(57) ABSTRACT

The disclosed invention relates to methods of modifying peptide compositions to increase stability and delivery efficiency. Specifically, the disclosed invention relates to methods to increase the stability and delivery efficiency of protein kinase C (PKC) modulatory peptide compositions. A “therapeutic peptide composition” comprises a “carrier peptide” and a “cargo peptide.” A “carrier peptide” is a peptide or amino acid sequence within a peptide that facilitates the cellular uptake of the therapeutic peptide composition. The “cargo peptide” is a PKC modulatory peptide. Peptide modifications to either the carrier peptide, the cargo peptide, or both, which are described herein increase the stability and delivery efficiency of therapeutic peptide compositions by reducing disulfide bond exchange, physical stability, reducing proteolytic degradation, and increasing efficiency of cellular uptake.

### Cross-Reference to Related Applications

This application is a continuation of U.S. application Ser. No. 17/749,004, filed May 19, 2022, which is a continuation of U.S. application Ser. No. 16/933,749, filed Jul. 20, 2020, which is a continuation of U.S. application Ser. No. 16/120,156, filed Aug. 31, 2018, which is a divisional of U.S. application Ser. No. 14/991,823, filed Jan. 8, 2016, now U.S. Pat. No. 10,077,438, which is a divisional of U.S. application Ser. No. 13/305,685, filed Nov. 28, 2011, now U.S. Pat. No. 9,255, 124 which is a divisional of U.S. application Ser. No. 12/017,985, filed Jan. 22, 2008, now U.S. Pat. No. 8,067,532, which claims the benefit of priority of U.S. Provisional Patent Application Nos. 60/881,419, filed Jan. 19, 2007 and 60/945,285, filed Jun. 20, 2007, all of which are incorporated herein by reference in their entirety.

### Reference to Sequence Listing

The instant application contains a Sequence Listing which is filed herewith electronically in XML format and is hereby incorporated by reference in its entirety. The XML copy, created on Feb. 28, 2024, is named 091508-0460-8007US07_SEQ.xml and is 273000 bytes in size.

### Technical Field

This application relates to compositions and methods to improve carrier of biologically active agents into cells in living tissue. The compositions and methods comprise a PKC modulatory peptide conjugated to a modified tat peptide that imparts improved plasma stability to the conjugate, allowing more efficient uptake of the PKC modulatory peptide into cells.

### Background Art

Research has produced many peptides that have potential as therapeutic compositions. Yet realizing and exploiting the full therapeutic potential of peptides directed against intracellular targets has yet to be achieved, for a variety of reasons. One of the most important of these is that most therapeutic peptides do not possess the ability to cross cell membranes to reach their therapeutic targets. One solution to this problem is the use of carrier peptides that act to ferry a cargo peptide into a target cell.

There are a number of notable examples of carrier peptides which are effective to facilitate the crossing of a target cell's membrane by a cargo peptide. One example is a peptide sequence derived from the TAT protein of the HIV virus. See U.S. Pat. No. 6,316,003, which is hereby incorporated by reference in its entirety. Another well-known carrier peptide sequence is the “poly-Arg” sequence. See, e.g., U.S. Pat. No. 6,306,993.

In many cases, the use of a disulfide bond to link the carrier and cargo peptides, producing the therapeutic peptide construct, is an effective strategy to solve the problem of targeting soluble peptides to intracellular targets. One theory explaining the usefulness of disulfide bonds holds that once the carrier-cargo construct enters a target cell, the two peptides can separate through disulfide bond reduction. This separation in the intracellular environment may allow a greater diffusion of cargo peptides within the cell as opposed to other linkage mechanisms which maintain the carrier-cargo link. With this said, however, the administration of therapeutic peptides still suffers from numerous challenges, such as disulfide bond exchange, proteolytic degradation and efficiency of cellular uptake. Methods directed to controlling these issues will increase the stability and potency of therapeutic peptides.

One way to increase the potency of a therapeutic peptide comprising a carrier peptide disulfide bonded to a cargo peptide is to reduce disulfide bond exchange. Disulfide bond exchange reduces the amount of a carrier-cargo peptide construct in a given sample by allowing a carrier peptide to exchange its cargo peptide for another carrier peptide, thus resulting in a carrier-carrier construct and a cargo-cargo construct. The carrier-only construct will have no therapeutic effect. The cargo-cargo construct will have a tremendously reduced, if not completely eliminated effect, since the carrier peptide enables the delivery of the cargo to its intracellular target. As such, the problem of controlling disulfide bond exchange is important to maximizing the therapeutic potential of a carrier-cargo peptide construct.

Another problem facing the use of therapeutic peptides is proteolytic degradation. Peptides are notoriously unstable molecules and frequently labile to proteolytic attack when administered to a subject. Labile carrier peptides which degrade upon administration will reduce or even eliminate the efficacy of the cargo peptide because the cargo depends upon the carrier peptide to reach the intracellular target. Thus, methods to control or eliminate the labile nature of therapeutic peptides are also important to maximizing a carrier-cargo peptide's therapeutic potential.

Increasing the efficiency of cellular uptake of a therapeutic peptide is yet another problem which can reduce the efficacy or potency of a therapeutic peptide. Optimization of carrier peptide sequences and placement relative to the cargo peptide provide methods for increasing the stability and potency of therapeutic peptide constructs.

### Disclosure of the Invention

The disclosed invention relates to methods of preparing a therapeutic peptide composition comprising a carrier peptide and a PKC activity modulating cargo peptide, whereby the resulting therapeutic peptide compositions have increased stability and potency relative to an unmodified therapeutic peptide. One embodiment of the invention is a method of decreasing disulfide bond exchange in a therapeutic peptide composition, comprising providing a therapeutic peptide composition, which comprises a carrier peptide comprising a first cysteine residue and a PKC activity modulating cargo peptide comprising a second cysteine residue, wherein the carrier peptide and the cargo peptide are linked by a cysteine-cysteine disulfide bond between the first and second cysteine residues, and introducing at least one aliphatic residue immediately proximate to the first or second cysteine residues, or both, whereby the rate of disulfide bond exchange is decreased relative to an unmodified therapeutic peptide composition.

Another embodiment of the disclosed invention related to a method of decreasing proteolytic degradation of a therapeutic peptide composition, comprising providing a therapeutic peptide composition, which comprises a carrier peptide and a PKC activity modulating cargo peptide, and wherein the carrier peptide is linked to the cargo peptide, identifying a proteolytically labile site on the carrier peptide, the cargo peptide, or both peptides, and modifying the amino acid sequence at the labile site such that the rate of proteolytic degradation at the site is decreased relative to an unmodified therapeutic peptide composition.

Another embodiment of the disclosed invention relates to a method of increasing plasma stability of a therapeutic peptide composition, comprising providing a therapeutic peptide composition, which comprises a carrier peptide and a PKC activity modulating cargo peptide, and wherein the carrier peptide is linked to the cargo peptide, modifying the amino terminal, carboxy terminal or both residues of the carrier peptide, the cargo peptide, or both, such that the plasma stability of the therapeutic peptide composition is increased relative to an unmodified therapeutic peptide composition.

Compositions are also contemplated disclosed herein. One embodiment of the disclosed compositions comprises a protein kinase C (PKC) modulatory peptide composition, comprising a PKC modulatory peptide covalently linked to an intracellular carrier peptide, wherein the intracellular carrier peptide, the modulatory peptide, or both are modified at the N-terminus.

### BRIEF DESCRIPTION OF THE DRAWINGS

FIG. 1 shows a graph plotting CK release against concentrations of therapeutic peptides KAI-9706 and KAI-1455.

FIG. 2 shows a graph plotting percent infarction against increasing concentrations of therapeutic peptides KAI-9706 and KAI-1455.

FIGS. 3A-3C show graphs plotting percent of intact therapeutic peptides KAI-9803, KAI-9706, and KAI-1455, surviving over time in human serum (FIG. 3A), pig serum (FIG. 3B), and rat serum (FIG. 3C).

FIGS. 4A and 4B show illustrate the conversion of a therapeutic peptide comprising a carrier peptide and a cargo peptide linked via a disulfide bond to a therapeutic peptide in linear form. This linear peptide (KP-01547) has been capped at its amino and carboxy termini and contains a short amino acid sequence linker.

FIG. 5 shows a graph plotting percent of intact therapeutic peptides (linear and non-linear) over time (days).

FIG. 6 shows a graph comparing the stability of therapeutic peptides over time (days).

FIG. 7 shows illustrated two linear peptides.

FIG. 8 shows a graph comparing the stability of various PKC-BI therapeutic peptides over time.

FIG. 9 shows a graph a graph comparing the stability of various PKC-Bu therapeutic peptides over time.

FIG. 10A-10D shows graphs illustrating the impact of temperature on the stability KAI-9706 at 26° C. (FIG. 10A) and 37° C. (FIG. 10B), and on KAI 1455 at 26° C. (FIG. 10C) and 37° C. (FIG. 10D).

FIG. 11 shows a graph plotting creatine kinase release in the presence of increasing concentrations of KAI-9803 or KAI-1355 peptides in a Langendorff in vitro post-ischemia assay, with 10 minutes perfusion.

FIG. 12 shows an illustration depicting the construction of KAI-1479.

FIGS. 13A and 13B show bar graphs of results from a reperfusion study, wherein 13A illustrates the time line of events during the experiment and 13B shows a bar graph illustrating the protective properties of various therapeutic peptides (KAI-9803, KAI-1479, and KAI-1482).

FIG. 14 shows the cargo peptide of SEQ ID NO:33 with a cysteine residue at the amino terminus of the peptide.

FIG. 15A-15D show four different possible configurations of therapeutic peptide multimers. FIG. 16 shows linear peptides KP-1680, KP-1681, KP-1633, and KP-1678.

FIG. 17A-17D show graphs of the percent of peptides remaining in test solutions over time. FIG. 17A shows the percent of peptides remaining in test solutions over time. FIG. 17B shows the percent of peptides remaining in test solutions over time. FIG. 17C shows the percent of peptides remaining in test solutions over time. FIG. 17D shows the percent of peptides remaining in test solutions over time.

### Modes of Carrying Out the Invention

The disclosed invention relates to methods of modifying peptide compositions to increase stability and delivery efficiency. Specifically, the disclosed invention relates to methods to increase the stability and delivery efficiency of protein kinase C (PKC) modulatory peptide compositions. A “therapeutic peptide composition” comprises a “carrier peptide” and a “cargo peptide.” A “carrier peptide” is a peptide or amino acid sequence within a peptide that facilitates the cellular uptake of the therapeutic peptide composition. The “cargo peptide” is a PKC modulatory peptide. Peptide modifications to either the carrier peptide, the cargo peptide, or both, which are described herein increase the stability and delivery efficiency of therapeutic peptide compositions by reducing disulfide bond exchange, physical stability, reducing proteolytic degradation, and increasing efficiency of cellular uptake.

### Disulfide Bond Exchange

A preferred embodiment of the disclosed therapeutic peptide compositions provides a cargo peptide coupled to a carrier peptide via a disulfide bond between two joining sulfur-containing residues, one in each peptide. The disulfide bond of this embodiment can be unstable whether the therapeutic peptide composition is in solution, lyophilized, precipitated, crystallized, or spray-dried, leading to carrier-cargo combinations to degrade to carrier-carrier compositions, which are inactive, and cargo-cargo compositions, which are also inactive and are frequently insoluble. The stability of the disclosed therapeutic peptide compositions is improved through the use of chemical modifications and by controlling the physical environment of the peptide compositions prior to use.

### Chemical Modifications

The joining sulfur-containing residue can be placed anywhere in the sequence of the carrier or cargo peptides. For example, a preferred embodiment of the disclosed therapeutic peptide composition typically has the joining sulfur-containing residue at the amino terminus of the carrier and cargo peptides. The joining sulfur-containing residues can be placed at the carboxy termini of the peptides, or alternatively at the amino terminus of peptide and at the carboxy terminus of the other peptide. Additionally, the joining sulfur-containing residue can be placed anywhere within the sequence of either or both of the peptides. Placing the joining sulfur-containing residue within the carrier peptide, the cargo peptide, or both has been observed to reduce the rate of disulfide bond exchange.

An example of chemical modifications useful to stabilize the disulfide bonds of the therapeutic peptide compositions involves optimizing the amino acid residue or residues immediately proximate to the sulfur-containing residues used to join the carrier and cargo peptide. A preferred method of stabilizing the disulfide bond involves placing an aliphatic residue immediately proximate to the sulfur-containing residue in the carrier and/or cargo peptides. Aliphatic residues include alanine, valine, leucine and isoleucine. Thus, when the joining sulfur-containing residue is placed at the amino terminus of a peptide, an aliphatic residue is placed at the penultimate amino terminal position of the peptide to reduce the rate of disulfide bond exchange. When the joining sulfur-containing residue is located at the carboxy terminus of a peptide, an aliphatic residue is placed at the penultimate carboxy terminal position of the peptide to reduce the rate of disulfide bond exchange. When the joining sulfur-containing residue is located within the sequence of a peptide, the aliphatic residue can be place at either the amino terminal or carboxy terminal side of the residue, or at both sides.

A variety of sulfur-containing residues are contemplated for use with the presently disclosed invention. Cysteine and cysteine analogs can also be used as the joining cysteine residues in the peptide composition. Particular cysteine analogs include D-cysteine, homocysteine, alpha-methyl cysteine, mercaptopropionic acid, mercaptoacetic acid, penicillamine, acetylated forms of those analogs capable of accepting an acetyl group, and cysteine analogs modified with other blocking groups. For example, the use of homocysteine, acetylated homocysteine, penicillamine, and acetylated penicillamine in the cargo, the carrier, or both peptides have been shown to stabilize the peptide composition and decrease disulfide bond exchange. Alpha-methyl cysteine inhibits disulfide degration because the base-mediated abstraction of the alpha hydrogen from one cysteine is prevented by the presence of the sulfur atom. Cargo/carrier peptide conjugates joined by disulfide bonds have been shown to be more resistant to glutathione reduction than unmodified peptides. Other cysteine analogs are also useful as joining cysteines. Similarly, stereoisomers of cysteine will inhibit disulfide bond exchange.

Disulfide bond exchange can be eliminated completely by linking the carrier and cargo peptides to form a single, linear peptide. This method is discussed below.

### Physical Stability

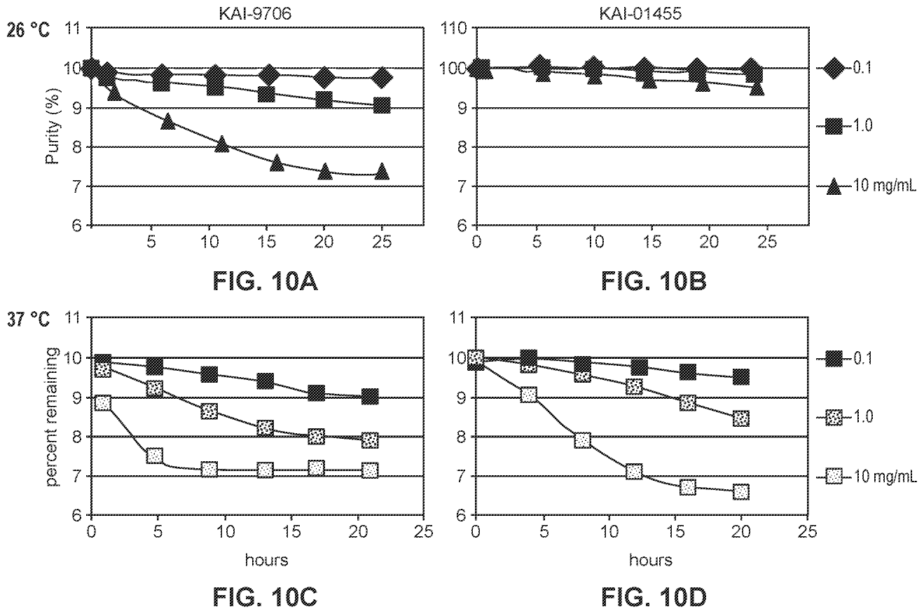

The physical environment of the disulfide has an effect on stability. As shown (in part) in FIGS. 10A-10D, stability increases in solution as the pH of the solution decreases (acidic environment better than basic), the temperature of the solution decreases, and as the concentration of the peptide composition in solution decreases. In the lyophilized form, stability increases as the pH decreases, the temperature decreases, and the ratio of the peptide composition to excipient increases. Preferred excipients are discussed in U.S. patent application Ser. No. 11/240,962, filed Sep. 30, 2005, which is hereby incorporated by reference in its entirety.

The unexpected “excipient effect” was most pronounced for mannitol, which is a highly crystalline excipient. Using less crystalline excipients (such as sucrose) or even using no excipient, showed much less dependency on peptide composition quantity. Although not wishing to be bound or limited by any theory, it is thought that use of a non-crystalline excipient creates an amorphous matrix, which helps prevent intermolecular associations. Theoretically, in a crystalline matrix the peptide composition is excluded and forced to the walls of the vial, perhaps causing high local concentrations. With low amount of API the resulting thin film has high peptide-glass contact area and the silica is destabilizing.

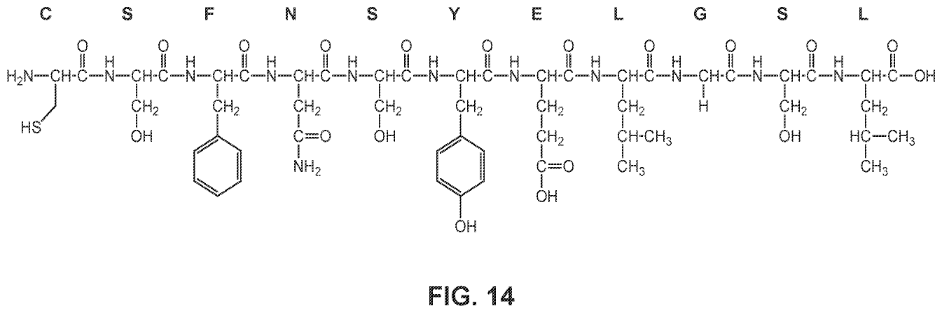

A number of factors impact the efficiency with which a therapeutic peptide composition is taken up by a target cell. For example, the solubility of a therapeutic peptide impacts the efficiency with which the peptide is taken up by a target cell. In turn, the amino acid sequence of a carrier or cargo peptide largely determines that solubility the peptide compositions in which they are used. Some peptides, particularly cargo peptides, will contain hydrophobic residues, (e.g., Phe, Tyr, Leu), with regular spacing which allows for intramolecular interactions by a “zipper” mechanism leading to aggregation. An example of such a potentially problematic peptide is shown in FIG. 14. The illustrated sequence is believed to form a beta-strand in the VI domain of δPKC. Such peptides have the tendency to form insoluble deposits.

The solubility of such peptides can be improved by making certain modifications to the cargo peptide sequence. For example, the introduction of solubilizing groups at amino and or carboxy termini or on internal residues, such as hydrating groups, like polyethylene glycol (PEG), highly charged groups, like quaternary ammonium salts, or bulky, branched chains of particular amino acid residues will improve the solubility of peptides like the one illustrated in FIG. 14. Additionally, those hydrophobic side chains that are shown not to be required for activity can be eliminated by deletion or substitution with a conservative or non-interfering residue, such as an alanine, glycine, or serine, thus improving the solubility of the peptides.

### Proteolytic Degradation: Plasma Stability

Blood and plasma contain proteases which can degrade the protein kinase C modulatory peptides disclosed herein or the carrier peptides which facilitate the cellular uptake of the peptide composition, or both. One method to decrease proteolytic degradation of the carrier or cargo peptides is to mask the targets of the proteases presented by the therapeutic peptide composition.

Once the therapeutic peptide enters the plasma of a subject, it become vulnerable to attack by peptidases. Strategies are provided which address peptide degradation caused by exopeptidases (any of a group of enzymes that hydrolyze peptide bonds formed by the terminal amino acids of peptide chains) or endopeptidases (any of a group of enzymes that hydrolyze peptide bonds within the long chains of protein molecules). Exopeptidases are enzymes that cleave amino acid residues from the amino or carboxy termini of a peptide or protein, and can cleave at specific or non-specific sites. Endopeptidases, which cleave within an amino acid sequence, can also be non-specific, however endopeptidases frequently recognize particular amino sequences (recognition sites) and cleaves the peptide at or near those sites.

One method of protecting peptide compositions from proteolytic degradation involves the “capping” the amino and/or carboxy termini of the peptides. The term “capping” refers to the introduction of a blocking group to the terminus of the peptide via a covalent modification. Suitable blocking groups serve to cap the termini of the peptides without decreasing the biological activity of the peptides. Acetylation of the amino termini of the described peptides is a preferred method of protecting the peptides from proteolytic degradation. Other capping moicties are possible. The selection of acylating moiety provides an opportunity to “cap” the peptide as well as adjust the hydrophobicity of the compound. For example, the hydrophobicity increases for the following acyl group series: formyl, acetyl, propanoyl, hexanoyl, myristoyl, and are also contemplated as capping moieties. Amidation of the carboxy termini of the described peptides is also a preferred method of protecting the peptides from proteolytic degradation.

Protecting peptides from endopeptidases typically involves identification and elimination of an endopeptidase recognition site from a peptide. Protease recognition cites are well known to those of ordinary skill in the art. Thus it is possible to identity a potential endoprotease recognition site and then eliminating that site by altering the amino acid sequence within the recognition site. Residues in the recognition sequence can be moved or removed to destroy the recognition site. Preferably, a conservative substitution is made with one or more of the amino acids which comprise an identified protease recognition site. The side chains of these amino acids possess a variety of chemical properties. For the purposes of the present discussion, the most common amino acids are categorized into 9 groups, listed below. Substitution within these groups is considered to be a conservative substitution.

Conservative Amino Acid Substitution

| | |
|---|---|
| Small/Aliphatic residues: | Gly, Ala, Val, Leu, Ile |
| Cyclic Imino Acid: | Pro |
| Hydroxyl Residues: | Ser, Thr |
| Acidic Residues: | Asp, Glu |
| Amide Residues: | Asn, Gin |
| Basic Residues: | Lys, Arg |
| Imidazole Residue: | His |
| Aromatic Residues: | Phe, Tyr, Trp |
| Sulfur-Containing Residues: | Met, Cys |

### Efficiency of Cellular Uptake

In addition to the modifications discussed above, improve utility for the disclosed therapeutic peptide compositions can be achieved by altering the linkage of the carrier and cargo peptides. For example, in one embodiment, carrier and cargo peptides are linked by a peptide bond to form a linear peptide. Stability and potency of the therapeutic peptides can also be increased through the construction of peptide multimers, wherein a plurality of cargo peptides is linked to one or more carrier peptides. An additional embodiment of the invention involving a cleavable linker sequence is also discussed.

### Linear Peptides

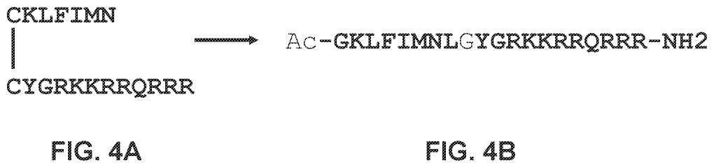

Another strategy to improve peptide composition stability involves joining the cargo and carrier peptides into a single peptide as opposed to joining the peptides via a disulfide bond. For example, in the embodiment shown in FIG. 4A, the cargo peptide (SEQ ID NO:13) linked via amino terminal cysteines. A linear version of the cargo and carrier peptides is shown in FIG. 4B, where the cargo and carrier peptides are linked via a short dipeptide linker (e.g., Ser-Gly). This linker is exemplary.

In the example illustrated, the C-terminus of cargo is linked to the N-terminus of the carrier via the linker. However, the other possible permutations are also contemplated, including linking the peptide via there C-termini, their N-termini, and where the carrier peptide is located at the N-terminal portion of the peptide composition.

Additionally, the steps discussed above to stabilize a disulfide bond linked peptide composition can also be used with a linear, where appropriate. For example, the linear peptide composition shown in FIG. 4B has been capped at both its amino and carboxy termini. Moreover sequences within the peptide can be scrambled or substituted with D-amino acids.

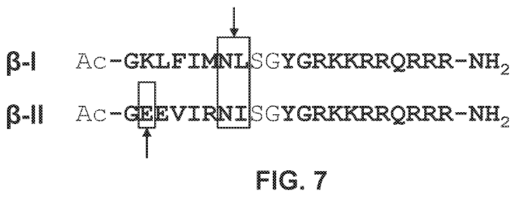

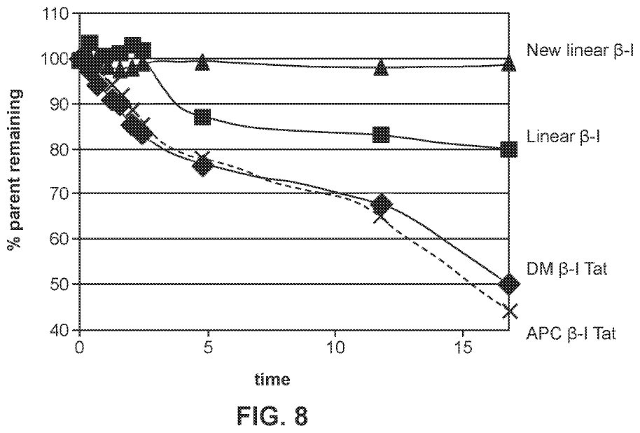

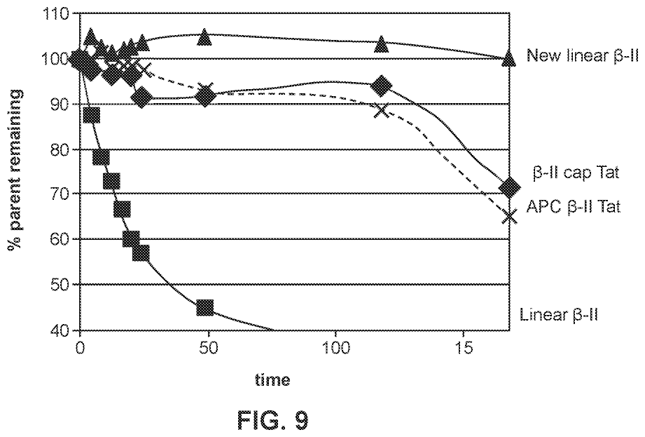

As shown in FIG. 7, deamination of Asn at position 7 of the β-I had been observed to cause significant instability in the linearized version of the peptide composition linked by Asn-Gly. Changing the Gly to Leu stabilized this linear peptide composition. Similarly, deamination of the Gln residue at position 2 of the linear β-II composition was observed to cause significant instability. Substitution with Glu improved stability of the linear composition. Data comparing the modified versions of these peptides is shown in FIGS. 8 and 9.

Without being limited to any particular theory, it is thought that deamination results from the attack of the alpha or main-chain amide HN-C-terminal to the Asn residue on the side-chain amide of Asn, generating the cyclic aspartamide intermediate which can hydrolyze to an aspartic acid moicty. Increasing the size of the residue C-terminal to Asn is thought to increase the steric hinderance on the main-chain amide, significantly slowing deamidation.

Peptide Multimers

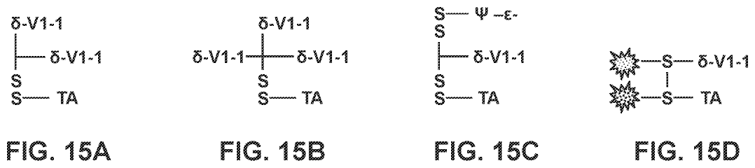

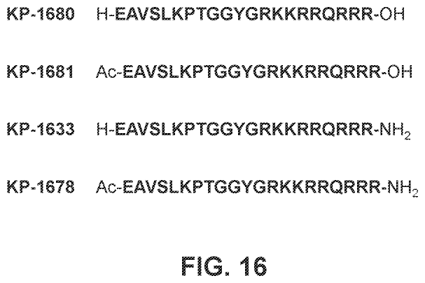

Another method of improving stability and potency is available by forming multimers with a plurality of cargo peptides associated with one or more carrier peptides. Examples of such formulations are shown in FIG. 15. Branched, multivalent peptide compositions will increase avidity, potency and stability of the compositions. By engineering cleavage sites or other release mechanisms into the multimer compositions, the multiple conjugates can release nearly simultaneously, PKC modulatory cargo peptides inside a target cell. An example of multimeric peptides is discussed in Yu et al. JBC 275(6):3943-9(2000).

### Cleavable Sequence

Typically the carrier and cargo are linked by a linkage that can be cleaved by ubiquitous enzymes such as esterases, amidases, and the like. It is assumed that the concentration of such enzymes is higher inside cells rather than in the extracellular milieu. Thus, once the conjugate is inside a cell, it is more likely to encounter an enzyme that can cleave the linkage between cargo and carrier. The enzyme can thus release the biologically active cargo inside a cell, where it presumably is most useful.

Protein Kinase C Modulatory Peptides

The term protein kinase C modulatory peptide refers to a peptide derived from a PKC isozyme-and/or variable region. Various PKC isozyme-and variable region-specific peptides have been described and can be used with the presently disclosed invention. Preferably, the PKC modulatory peptide is a V1, V3 or V5-derived peptide. (The terminology “V1” and “C2” are synonymous.) The following US Patents or Patent Applications describe a variety of suitable peptides that can be used with the presently disclosed invention: 5,783,405, 6,165,977, 6,855,693, US2004/0204364, US2002/0150984, US2002/0168354, US2002/057413, US2003/0223981, US2004/0009922 and 10/428,280, each of which are incorporated herein by reference in their entirety. Table 1 provides a listing of preferred PKC modulatory peptides for use with the present invention.

TABLE 1
Cargo Peptides derived from PKC isozymes

| Peptide | SEQ ID NO. | Sequence |
|---|---|---|
| αV3-1 | SEQ ID NO: 2 | I-P-E-G-D-E-E-G |
| αV5-1 | SEQ ID NO: 3 | Q-L-V-I-A-N |
| αV5-1.1 | SEQ ID NO: 4 | G-L-G-A-E-N |
| αV5-1.2 | SEQ ID NO: 5 | A-R-G-A-E-N |
| αV5-1.3 | SEQ ID NO: 6 | C-G-K-G-A-E-N |
| αV5-1.4 | SEQ ID NO: 7 | C-G-K-G-A-E-N |
| βC2-1 | SEQ ID NO: 8 | K-Q-K-T-K-T-I-K |
| βC2-2 | SEQ ID NO: 9 | M-D-P-N-G-L-S-D-P-Y-V-K-L |
| βC2-3 | SEQ ID NO: 10 | I-P-D-P-K-S-E |
| βC2-4 | SEQ ID NO: 11 | S-L-N-P-E-W-N-E-T |
| βV3-1 | SEQ ID NO: 12 | V-P-P-E-G-S-E-A |
| βIV5-1 | SEQ ID NO: 13 | K-L-F-I-M-N |
| βIV5-2 | SEQ ID NO: 14 | R-D-K-R-D-T-S |
| βIV5-2.1 | SEQ ID NO: 15 | C-A-R-D-K-R-D-T-S |
| βIV5-2.2 | SEQ ID NO: 16 | G-R-D-K-R-D-T-S |
| βIV5-2.3 | SEQ ID NO: 17 | A-R-D-K-R-D-T-S |
| βIV5-3 | SEQ ID NO: 18 | A-R-D-K-R-D-T-S-N-F-D-K |
| βIV5-4 | SEQ ID NO: 19 | A-G-F-S-Y-T-N-P-E-F-V-I-N-V |
| βIIV5-1 | SEQ ID NO: 20 | Q-E-V-I-R-N |
| βIIV5-2 | SEQ ID NO: 21 | C-G-R-N-A-E |
| βIIV5-3 | SEQ ID NO: 22 | A-C-G-R-N-A-E |
| βIIV5-3.1 | SEQ ID NO: 23 | A-C-G-K-N-A-E |
| βIIV5-4 | SEQ ID NO: 24 | K-A-C-G-R-N-A-E |
| βIIV5-5 | SEQ ID NO: 25 | C-G-R-N-A-E-N |
| βIIV5-6 | SEQ ID NO: 26 | A-C-G-R-N-A-E |
| βIIV5-7 | SEQ ID NO: 27 | S-F-V-N-S-E-F-L-K-P-E-V-L-S |
| γV3-1 | SEQ ID NO: 28 | V-A-D-A-D-N-C-S |
| γV5-1 | SEQ ID NO: 29 | G-R-S-G-E-N |
| γV5-1.1 | SEQ ID NO: 30 | G-L-S-G-E-N |
| γV5-2 | SEQ ID NO: 31 | R-L-V-L-A-S |
| γV5-3 | SEQ ID NO: 32 | P-C-G-R-S-G-E-N |
| δV1-1 | SEQ ID NO: 33 | C-S-F-N-S-Y-E-L-G-S-L |
| Leu-Truncate | SEQ ID NO: 165 | C-S-F-N-S-Y-E-L-G-S |
| δV1-1.1 | SEQ ID NO: 34 | S-F-N-S-Y-E-L-G-S-L |
| δV1-1.2 | SEQ ID NO: 35 | T-F-N-S-Y-E-L-G-S-L |
| δV1-1.3 | SEQ ID NO: 36 | A-F-N-S-N-Y-E-L-G-S-L |
| δV1-1.4 | SEQ ID NO: 37 | S-F-N-S-Y-E-L-G-T-L |
| δV1-1.5 | SEQ ID NO: 38 | S-T-N-S-Y-E-L-G-S-L |
| δV1-1.6 | SEQ ID NO: 39 | S-F-N-S-F-E-L-G-S-L |
| δV1-1.7 | SEQ ID NO: 40 | S-N-S-Y-D-L-G-S-L |
| δV1-1.8 | SEQ ID NO: 41 | S-F-N-S-Y-E-L-P-S-L |
| δV1-1.9 | SEQ ID NO: 42 | T-F-N-S-Y-E-L-G-T-L |
| δV1-1.10 | SEQ ID NO: 43 | S-F-N-S-Y-E-I-G-S-V |
| δV1-1.11 | SEQ ID NO: 44 | S-F-N-S-Y-E-V-G-S-I- |
| δV1-1.12 | SEQ ID NO: 45 | S-F-N-S-Y-E-L-G-S-V |
| δV1-1.13 | SEQ ID NO: 46 | S-F-N-S-Y-E-L-G-S-I |
| δV1-1.14 | SEQ ID NO: 47 | S-F-N-S-Y-E-I-G-S-L |
| δV1-1.15 | SEQ ID NO: 48 | S-F-N-S-Y-E-V-G-S-L |
| δV1-1.16 | SEQ ID NO: 49 | A-F-N-S-Y-E-L-G-S-L |
| δV1-1.17 | SEQ ID NO: 50 | Y-D-L-G-S-L |
| δV1-1.18 | SEQ ID NO: 51 | F-D-L-G-S-L |
| δV1-1.19 | SEQ ID NO: 52 | Y-D-I-G-S-L |
| δV1-1.20 | SEQ ID NO: 53 | Y-D-V-G-S-L |
| δV1-1.21 | SEQ ID NO: 54 | Y-D-L-P-S-L |
| δV1-1.22 | SEQ ID NO: 55 | Y-D-L-G-L-L |
| δV1-1.23 | SEQ ID NO: 56 | Y-D-L-G-S-I |
| δV1-1.24 | SEQ ID NO: 57 | Y-D-L-G-S-V |
| δV1-1.25 | SEQ ID NO: 58 | I-G-S-L |
| δV1-1.26 | SEQ ID NO: 59 | V-G-S-L |
| δV1-1.27 | SEQ ID NO: 60 | L-P-S-L |
| δV1-1.28 | SEQ ID NO: 61 | L-G-L-L |
| δV1-1.29 | SEQ ID NO: 62 | L-G-S-I |
| δV1-1.30 | SEQ ID NO: 63 | L-G-S-V |
| δV1-2 | SEQ ID NO: 64 | A-L-S-T-E-R-G-K-T-L-V |
| δV1-2.1 | SEQ ID NO: 65 | A-L-S-T-D-R-G-K-T-L-V |
| δV1-2.2 | SEQ ID NO: 66 | A-L-T-S-D-R-G-K-T-L-V |
| δV1-2.3 | SEQ ID NO: 67 | A-L-T-T-D-R-G-K-S-L-V |
| δV1-2.4 | SEQ ID NO: 68 | A-L-T-T-D-R-P-K-T-L-V |
| δV1-2.5 | SEQ ID NO: 69 | A-L-T-T-D-R-G-R-T-L-V |
| δV1-2.6 | SEQ ID NO: 70 | A-L-T-T-D-K-G-K-T-L-V |
| δV1-2.7 | SEQ ID NO: 71 | A-L-T-T-D-K-G-K-T-L-V |
| δV1-3 | SEQ ID NO: 72 | V-L-M-R-A-A-E-E-P-V |
| δV1-4 | SEQ ID NO: 73 | Q-S-M-R-S-E-D-E-A-K |
| δV1-5 | SEQ ID NO: 163 | A-F-N-S-Y-E-L-G-S |
| δv3-1 | SEQ ID NO: 74 | Q-G-F-E-K-K-T-G-V |
| δv3-2 | SEQ ID NO: 75 | D-N-N-G-T-Y-G-K-I |
| δv5-1 | SEQ ID NO: 76 | K-N-L-I-D-S |
| δv5-2 | SEQ ID NO: 77 | V-K-S-P-R-D-Y-S |
| δv5-2.1 | SEQ ID NO: 78 | V-K-S-P-C-R-D-Y-S |
| δv5-2.2 | SEQ ID NO: 79 | I-K-S-P-R-L-Y-S |
| δv5-3 | SEQ ID NO: 80 | K-N-L-I-D-S |
| δv5-4 | SEQ ID NO: 81 | P-K-V-K-S-P-R-D-Y-S-N |
| εV1-1 | SEQ ID NO: 82 | N-G-L-L-K-I-K |
| εV1-2 | SEQ ID NO: 83 | E-A-V-S-L-K-P-T |
| εV1-3 | SEQ ID NO: 84 | L-A-V-F-H-D-A-P-I-G-Y |
| εV1-4 | SEQ ID NO: 85 | D-D-F-V-A-N-C-T-I |
| εV1-5 | SEQ ID NO: 86 | W-I-D-L-E-P-E-G-R-V |
| εV1-6 | SEQ ID NO: 87 | H-A-V-G-P-R-P-Q-T-F |
| εV1-7 | SEQ ID NO: 88 | N-G-S-R-H-F-E-D |
| εV1-7.1 | SEQ ID NO: 89 | H-D-A-P-I-G-Y-D |
| εV1-7.2 | SEQ ID NO: 90 | H-D-A-P-I-G |
| εV1-7.3 | SEQ ID NO: 91 | H-D-A-A-I-G-Y-D |
| εV1-7.4 | SEQ ID NO: 92 | H-D-A-P-I-P-Y-D |
| εV1-7.5 | SEQ ID NO: 93 | H-N-A-P-I-G-Y-D |
| εV1-7.6 | SEQ ID NO: 94 | H-A-A-P-I-G-Y-D |
| εV1-7.7 | SEQ ID NO: 95 | A-D-A-P-I-G-Y-D |
| εV1-7.8 | SEQ ID NO: 96 | H-D-A-P-A-G-Y-D |
| εV1-7.9 | SEQ ID NO: 97 | H-D-A-P-I-G-A-D |
| εV1-7.10 | SEQ ID NO: 98 | H-D-A-P-I-A-Y-D |
| εV1-7.11 | SEQ ID NO: 99 | H-D-A-P-I-G-Y-A |
| εV3-1 | SEQ ID NO: 100 | S-S-P-S-E-E-D-R-S |
| εV3-2 | SEQ ID NO: 101 | P-C-D-Q-E-I-K-E |
| εV3-3 | SEQ ID NO: 102 | E-N-N-I-R-K-A-L-S |
| εV3-4 | SEQ ID NO: 103 | G-E-V-R-Q-G-Q-A |
| εV5-1 | SEQ ID NO: 104 | E-A-V-K-Q |
| εV5-2 | SEQ ID NO: 105 | I-K-T-K-R-D-V |
| εV5-2.1 | SEQ ID NO: 106 | I-K-T-K-R-L-I |
| εV5-3 | SEQ ID NO: 107 | C-E-A-I-V-K-Q |
| εV5-4 | SEQ ID NO: 108 | T-K-R-D-V-N-N-F-D-Q |
| ζv1-1 | SEQ ID NO: 109 | V-R-L-K-A-H-Y |
| ζv1-2 | SEQ ID NO: 110 | V-D-S-E-G-D |
| ζv1-3 | SEQ ID NO: 111 | V-F-P-S-I-P-E-Q |
| ζv3-1 | SEQ ID NO: 112 | S-Q-E-P-P-V-D-D-K-N-E-D-A-D-L |
| ζv3-2 | SEQ ID NO: 113 | I-K-D-D-S-E-D |
| ζv3-3 | SEQ ID NO: 114 | P-V-I-D-G-M-D-G-I |
| ζv5-1 | SEQ ID NO: 115 | E-D-A-I-K-R |
| ζv5-1.1 | SEQ ID NO: 116 | E-D-A-I-R |
| ζv5-2 | SEQ ID NO: 117 | I-T-D-D-Y-G-L-D |
| ζv5-2.1 | SEQ ID NO: 118 | I-T-D-D-Y-G-D-L |
| ζv5-3 | SEQ ID NO: 119 | D-D-Y-G-L-D-N |
| ηV1-1 | SEQ ID NO: 120 | N-G-Y-L-R-V-R |
| ηV1-2 | SEQ ID NO: 121 | E-A-V-G-L-Q-P-T |
| ηV1-3 | SEQ ID NO: 122 | L-A-V-F-H-E-T-P-L-G-Y |
| ηV1-4 | SEQ ID NO: 123 | D-F-V-A-N-C-T-L |
| ηV1-5 | SEQ ID NO: 124 | W-V-D-L-E-P-E-G-K-V |
| ηV1-6 | SEQ ID NO: 125 | H-S-L-F-K-K-G-H |
| ηV1-7 | SEQ ID NO: 126 | T-G-A-S-D-T-F-E-G |
| ηV5-1 | SEQ ID NO: 127 | E-G-H-L-P-M |
| ηV5-1.1 | SEQ ID NO: 128 | E-G-H-D-P-M |
| ηV5-2 | SEQ ID NO: 129 | I-K-S-R-E-D-V-S |
| ηV5-3 | SEQ ID NO: 130 | V-R-S-R-E-D-V-S |
| ηV5-4 | SEQ ID NO: 131 | P-R-I-K-S-R-E-D-V |
| λv1-1 | SEQ ID NO: 132 | H-Q-V-R-V-K-A-Y-Y-R |
| λv1-2 | SEQ ID NO: 133 | Y-E-L-N-K-D-S-E-L-L-I |
| λv3-1 | SEQ ID NO: 134 | M-D-Q-S-S-M-H-S-D-H-A-Q-T-V-I |
| λv3-2 | SEQ ID NO: 135 | L-D-Q-V-G-E-E |
| λv3-3 | SEQ ID NO: 136 | E-A-M-N-T-R-E-S-G |
| λv5-1 | SEQ ID NO: 137 | D-D-I-V-R-K |
| μV5-2 | SEQ ID NO: 138 | V-K-L-C-D-F-G-F |
| μV5-2.1 | SEQ ID NO: 139 | I-R-L-C-D-F-A-F |
| μV5-3 | SEQ ID NO: 140 | Q-V-K-L-C-D-F-G-F-A |
| μV1-1 | SEQ ID NO: 141 | M-S-V-P-P-L-L-R-P |
| μV1-2 | SEQ ID NO: 142 | K-F-P-E-C-G-F-Y-G-L-Y |
| μV3-1 | SEQ ID NO: 143 | D-P-D-A-D-Q-E-D-S |
| μV3-2 | SEQ ID NO: 144 | S-K-D-T-L-R-K-R-H |
| μV3-3 | SEQ ID NO: 145 | I-T-L-F-Q-N-D-T-G |
| μV3-4 | SEQ ID NO: 146 | G-S-N-S-H-K-D-I-S |
| μV5-1 | SEQ ID NO: 147 | S-D-S-P-E-A |
| ΘV1-1 | SEQ ID NO: 148 | G-L-S-N-F-D-C-G |
| ΘV1-2 | SEQ ID NO: 149 | Y-V-E-S-E-N-G-Q-M-Y-I |
| ΘV1-3 | SEQ ID NO: 150 | I-V-K-G-K-N-V-D-L-I |
| ΘV1-4 | SEQ ID NO: 151 | D-M-N-E-F-E-T-E-G-F |
| ΘV3-1 | SEQ ID NO: 152 | C-S-I-K-N-E-A-R-L |
| ΘV3-2 | SEQ ID NO: 153 | G-K-R-E-P-Q-G-I-S |
| ΘV3-3 | SEQ ID NO: 154 | D-E-V-D-K-M-C-H-L |
| ΘV5-1 | SEQ ID NO: 155 | R-A-L-I-N-S |
| ΘV5-2 | SEQ ID NO: 156 | V-K-S-P-F-D-C-S |
| ΘV5-2.1 | SEQ ID NO: 157 | V-R-S-P-F-D-C-S |
| ΘV5-3 | SEQ ID NO: 158 | D-R-A-L-I-N-S |
| ιV5-1 | SEQ ID NO: 159 | I-S-G-E-F-G-L-D |
| ιV5-1.1 | SEQ ID NO: 160 | C-S-G-E-F-G-L-D |
| ιV5-2 | SEQ ID NO: 161 | D-D-D-I-V-R-K |
| ιV5-3 | SEQ ID NO: 162 | D-D-I-V-R-K |

TABLE 2
Carrier Peptides

| | | |
|---|---|---|
| TAT Carrier | SEQ ID NO: 166 | YGRKKRRQRRR |
| Peptide | | |
| TAT Carrier | SEQ ID NO: 164 | CYGRKKRRQRRR |
| Peptide with N- | | |
| terminal Cys | | |

Other examples of carries include octa-Arg, octa-D-Arg, and Antennapedia derived peptides, which are known in the art.

The following examples are offered to illustrate but not to limit the invention.

### EXAMPLE 1

Exopeptidase protection: Plasma Stability of Capped Peptides

Plasma stability of capped peptides was compared. KAI-9706 was modified stepwise at its amino and carboxy termini. Plasma stability as measured by the percent of peptide composition remaining after 15 minutes. The results are provided in Table 2.

TABLE 2
Plasma Stability of KAI-9706

| | | cargo | | | |
|---|---|---|---|---|---|
| | | H—OH | Ac—OH | H—NH₂ | Ac—NH₂ |
| carrier | H—OH | 1 | 1 | 0 | 0 |
| | Ac—OH | 57 | nd | nd | 48 |
| | H—NH₂ | 60 | nd | nd | 51 |
| | Ac—NH₂ | 92 | 93 | 90 | 90 |

% parent remaining at 15 mins
t_{1/2} in rat plasma = 40-45 mins for longest-lived derivatives

The data provided above shows that the peptide composition, comprising unmodified cargo and carrier peptides, was the least stable. Moreover, protection of the carrier peptide alone failed to increase the half life of the peptide composition in plasma. Moreover, modification of the cargo peptide with the carrier peptide unmodified had no apparent effect on half-life stability in plasma. However, the addition of protecting groups to the carrier peptide, whether at the amino or carboxy termini lead to a marked and nearly equivalent increase in plasma stability for the peptide composition. Protection of both groups in the carrier peptide provided additional protection. Interestingly, protection of the cargo peptide had little or no effect on the stability of the composition.

### EXAMPLE 2

### Effect of D-Peptides on Plasma Stability

KAI-9706 was engineered with D-amino acids to determine their impact on peptide composition stability. Unmodified KAI-9706 was compared to a peptide composition with the same amino acid sequence, however the amino acids used were d-enantiomers instead of 1-amino acids. A retro-inverso version and a scrambled version of the peptide composition were also prepared. The data from the experiment is shown in Table 3.

TABLE 3
Plasma Stability of KAI-9706

| | | cargo | | | |
|---|---|---|---|---|---|
| | | All-L | All-D | scrambled | R/I |
| carrier | All-L | 1 | 0 | 2 | |
| | All-D | 88 | 100 | 67 | |
| | R/I | | | | 100 |

% parent remaining at 15 mins

Modification of the carrier showed the great propensity in improving the half life of the composition while modification of the cargo showed little effect.

### EXAMPLE 3

### Capped KAI-9706 Maintains In Vitro Potency

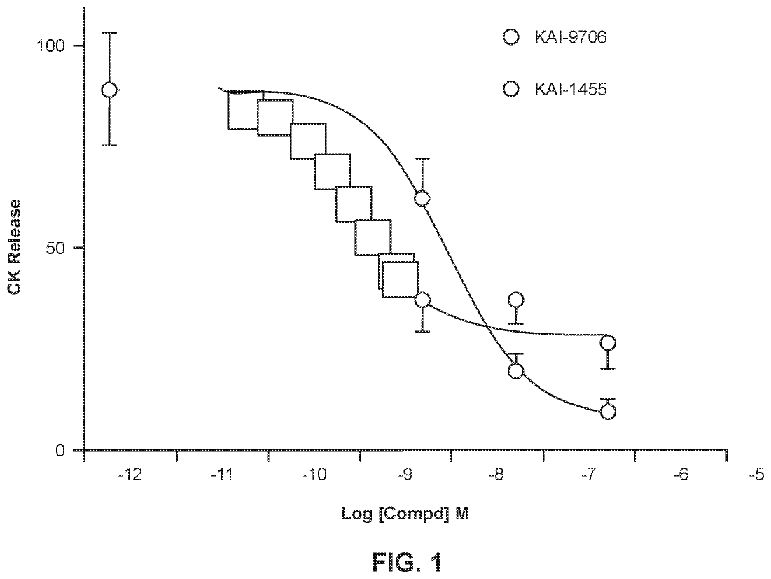

Capping the carrier peptide portion KAI-9706 (KAI-1455) was shown to increase the plasma half life of the peptide composition. The ability of the capped composition to inhibit ischemic damage in a rat heat model (Langendorff Assay) was evaluated versus the uncapped form. The results are shown in FIG. 1.

### EXAMPLE 4

### Capped KAI-9706 Shows Increased Potency

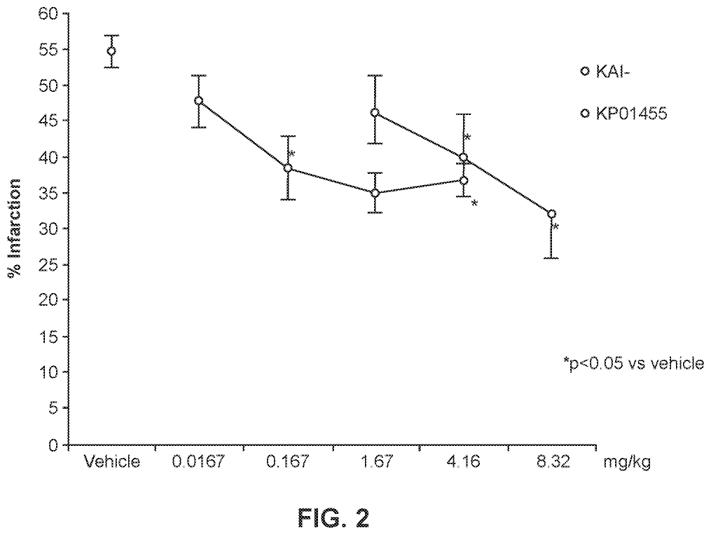

KAI-1455 was tested in a stroke model. As shown in FIG. 2, the capped version of the peptide composition provided increased protection to brain tissue as judged by percent infract. This data shows that significant protection of brain tissue was achieved at lower doses.

### EXAMPLE 5

### Peptide Stability is Increased Regardless of Species

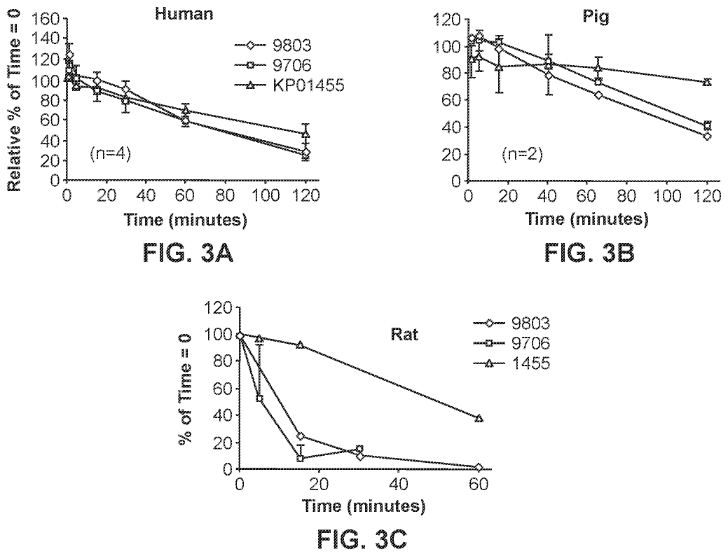

The stability of modified KAI-9706 peptide (KAI-1455) was compared against KAI-9706 and KAI-9803 in human (FIG. 3A), pig (FIG. 3B) and rat serum (FIG. 3C). The capped version, KAI-1455, showed increased plasma stability in all three species.

### EXAMPLE 6

### Capped KAI-9706 Shows Increased Potency

KAI-1455 was tested in a stroke model. As shown in FIG. 2, the capped version of the peptide composition provided increased protection to brain tissue as judged by percent infract. This data shows that significant protection of brain tissue was achieved at lower doses.

### EXAMPLE 7

### Peptide Stability is Increased Regardless of Species

The stability of modified KAI-9706 peptide (KAI-1455) was compared against KAI-9706 and KAI-9803 in human (FIG. 3A), pig (FIG. 3B) and rat serum (FIG. 3C). The capped version, KAI-1455, showed increased plasma stability in all three species.

### EXAMPLE 8

### Stability of Linear Peptides

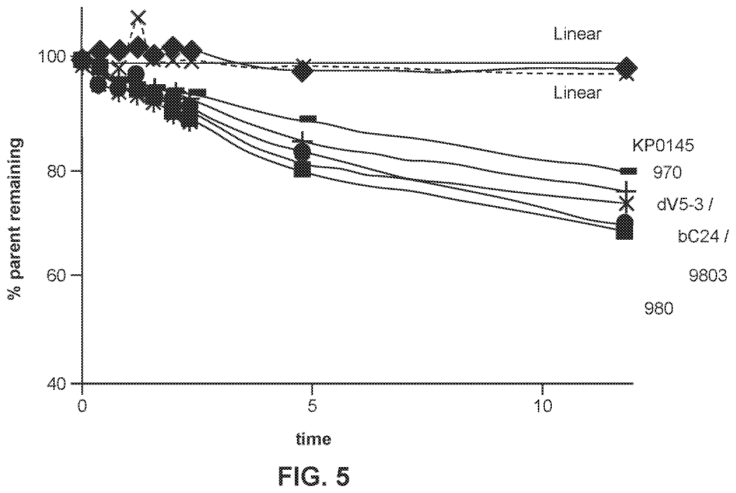

Linear versions of KAI-9803 and BC2-4 were constructed to evaluate their stability relative to disulfide bond linked versions of these and other peptide compositions. The peptides were placed in solution at 0.1 mg/ml in PBS (pH 7.4) at 37° C. As shown in FIG. 5, the linear versions of KAI-9803and BV2-4 showed increased stability.

### EXAMPLE 9

### Linear PKC-β_{I} and PKC-β_{II} Peptide Compositions Show Increased Stability Over Disulfide inked Compositions

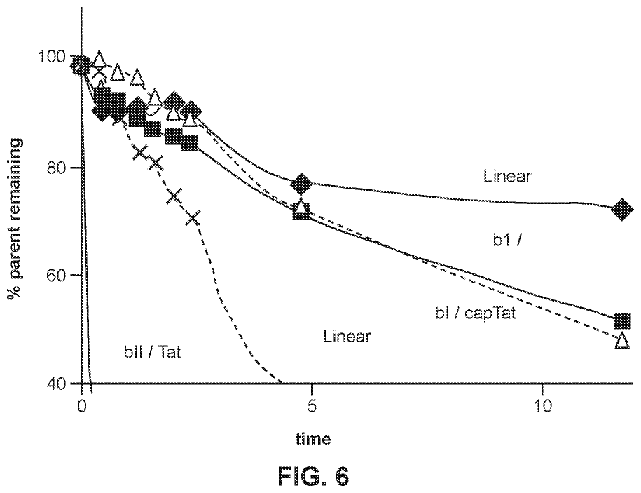

Linear and disulfide bond linked versions of PKC-β_{I} and PKC-β_{II} peptide compositions were incubated under the conditions described in Example 8. As can be seen in FIG. 6, the linear forms of the PKC-β_{I} and PKC-β_{II} peptides showed increased stability as compared to their non-linear counterparts.

### EXAMPLE 10

Improved Stability of Linear PKC-β_{I} and PKC-β_{II} Peptide Compositions

The linear versions of PKC-β_{I} and PKC-β_{II} peptide compositions showed improved stability but were also the subject of deamination reactions. In particular, the Asn residues at position 7 of the PKC-βI and PKC-βII peptides and the Gln at position 2 of the PKC-βII peptide. These linear peptide compositions were modified by substituting the Gly immediately C-terminal to the Asn with either Leu in the βI peptide composition or Gly to Ile in the βII peptide composition. The Gln at position 2 of the βII peptide composition was also substituted with a Glu residue. The stability of the peptides was studied under the conditions described in Example 8. As shown in FIGS. 8 and 9, the amino acid substitutions discussed above served to stabilize these linear peptide compositions.

### EXAMPLE 11

### KAI-9803 Derivative (KAI-1355) Maintains Potency

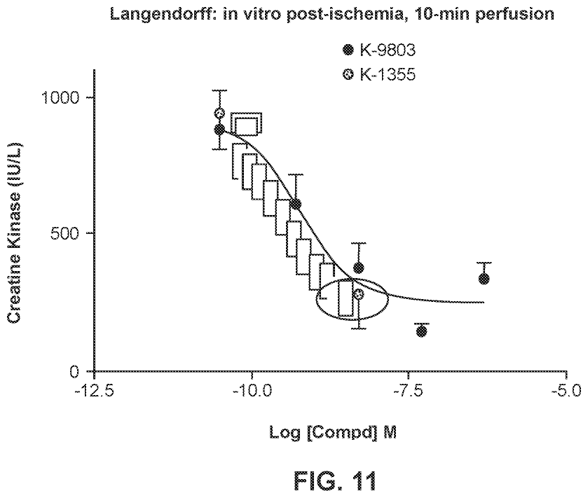

A truncated version of KAI-9803, KAI-1355, in which the carboxy terminal leucine was removed was tested for potency. Stability studies with KAI-1355 showed that deletion of the C-terminal Leu residue increased the stability of this cargo peptide. Potency of the derivative peptide composition was compared to that of the full length version, KAI-9803 in a Langendorff in vitro post-ischemia model. The results of the experiment are shown in FIG. 11. As shown, KAI-1355 (the modified version of KAI-9803) is still capable of protecting cardiac tissue from ischemia with potency comparable to that of the full length KAI-9803.

### EXAMPLE 12

### Optimization of KAI-9803 to Produce KAI-1479

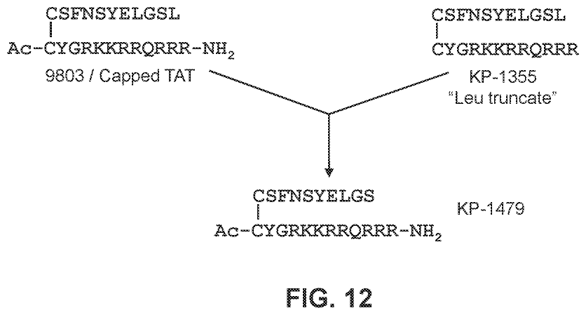

Having demonstrated that truncation of the cargo peptide ofKAI-9803 had little or now effect on potency, while stabilizing the peptide composition. As illustrated in FIG. 12, a capped version of the TAT carrier peptide was bound to the truncated cargo peptide of KAI-1355, producing KAI-1479, which comprises a truncated 9803 cargo peptide and a fully capped TAT carrier peptide.

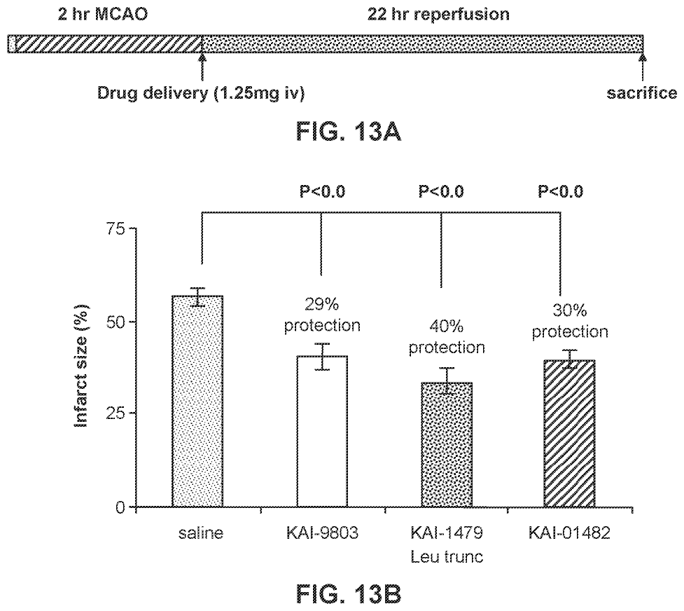

The modified KAI-1479, KAI-9803 and KAI-1482 peptide compositions were assayed in a rat middle cerebral artery occlusion (MCAO) stroke model to determine the ability of the peptide compositions to inhibit infarct size. The rats were subjected to a 2 hour period of cerebral arterial occlusion. Each of the peptide compositions or saline was administered to the test animals immediately prior to a 22 hour reperfusion period, after which time the animals were sacrificed and the infarct size was measured. As shown in FIG. 13, the modified KAI-1479 peptide composition showed an increased ability to retard infarct size as compared to KAI-9803. KP-01482 has a cargo sequence (CELGSLQAEDD) linked to a TAT peptide with a N-Term Cys, which is capped at both ends and disulfide conjugated to the cargo.

### EXAMPLE 13

In Vitro Biological Stability of a Series of Linear Epsilon PKC Inhibitors

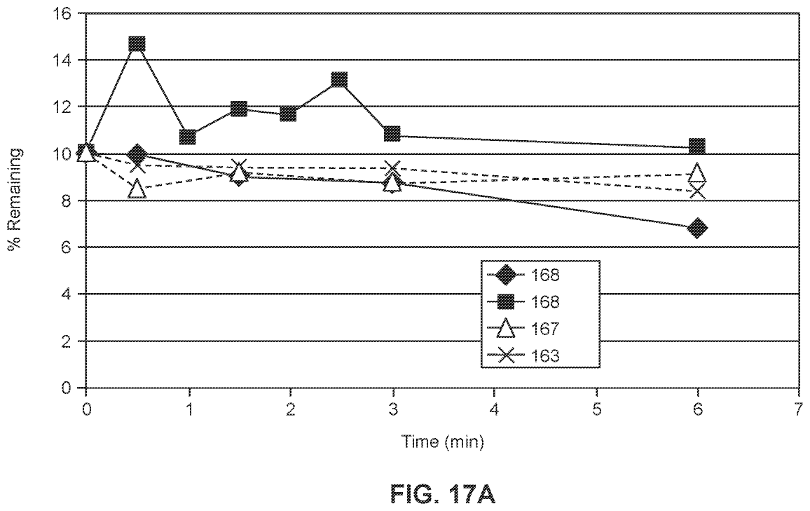

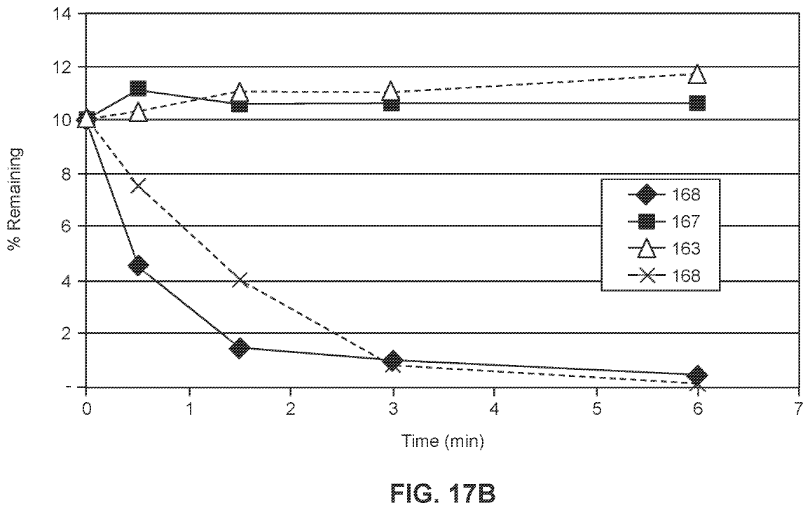

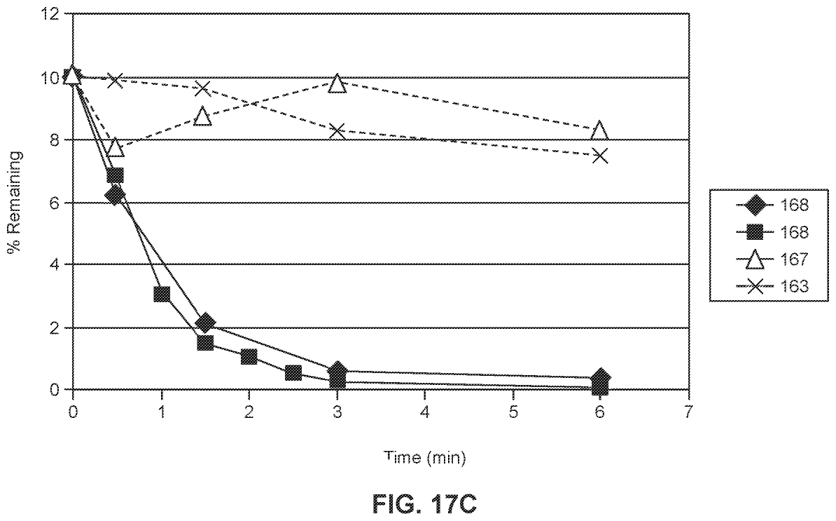

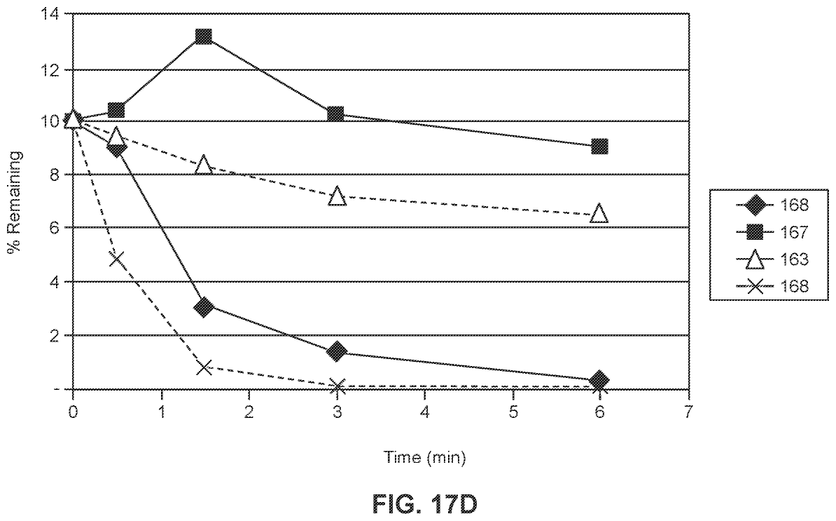

The effect of N-terminal acetylation and C-terminal amidation on compound stability in plasma and serum from rat and human was studied. The linear peptides examined are shown in FIG. 16. The compounds were tested in the plasma/serum at a concentration of 100 μg/ml. The solutions were incubated at room temperature, precipitated with 5% TCA, and then the supernatant was neutralized with ammonium acetate. The peptides were then analyzed by LC/MS. As can be seen from the data in FIGS. 17A-17D, all the tested compounds were relatively stable in human plasma but KP-1633 and KP-1678, containing C-terminal amides, showed increased stability in human serum. N-terminal acetylation alone did not stabilize the peptides. Interestingly, the amino acid sequence of KP-1680 and its degradation products indicated that the metabolized forms of the peptide showed sequential cleavage of arginine residues from the C-terminus. Carboxypeptidase N activity in the serum but not the plasma could account for the difference in observed stability. The plasma samples were collected with EDTA, which is known to inhibit this zinc metalloprotease.

We claim:

1. A protein kinase C (PKC) modulatory peptide conjugate, comprising:
a PKC modulatory peptide covalently linked to an intracellular carrier peptide, wherein the intracellular carrier peptide, the modulatory peptide, or both are modified at the N-terminus, and
a pharmaceutically acceptable excipient.

2. The conjugate of claim 1, wherein the PKC modulatory peptide is an inhibitory peptide which inhibits activity of a PKC isozyme or an activator peptide which promotes activity of a PKC isozyme.

3. The conjugate of claim 1, wherein the intracellular carrier peptide is a modified tat peptide comprising YGRKKRRQRRR (SEQ ID NO: 166) or a modified tat peptide comprising CYGRKKRRQRRR (SEQ ID NO: 164).

4. The conjugate of claim 3, wherein the modified tat peptide is substituted at its N-terminal end by an acyl, alkyl, or sulfonyl group.

5. The conjugate of claim 4, wherein the modified tat peptide is acylated at its N-terminal end.

6. The conjugate of claim 1, wherein the tat peptide is further modified at its C-terminal end.

7. The conjugate of claim 1, wherein the tat peptide is further modified by formation of an amide at its C-terminal end.

8. The conjugate of claim 1, wherein the PKC modulatory peptide is covalently linked to a side chain of an amino acid of the modified tat peptide.

9. The conjugate of claim 1, wherein the PKC modulatory peptide is covalently linked to a side chain of a residue selected from cysteine, lysine, and tyrosine.

10. The conjugate of claim 3, wherein the PKC modulatory peptide is covalently linked to a side chain of the N-terminal cysteine residue of the modified tat peptide comprising CYGRKKRRQRRR (SEQ ID NO: 164).

11. The conjugate of claim 10, wherein the N-terminal cysteine of the tat peptide is acylated.

12. The conjugate of claim 10, wherein the C-terminal arginine of the tat peptide is a primary carboxamide.

13. The conjugate of claim 10, wherein the PKC modulatory peptide is modified by either acylation at its N-terminal end, or amidation at its C-terminal end, or by both acylation at its N-terminal end and amidation at its C-terminal end.

14. The conjugate of claim 10, wherein the PKC modulatory peptide is covalently linked to the tat peptide through the sulfhydryl group of the cysteine residue of the tat peptide.

15. The conjugate of claim 1, which further comprises a second carrier peptide.

16. A linear therapeutic peptide, comprising:
a carrier peptide and a PKC activity modulating cargo peptide, wherein the carrier peptide and the cargo peptide are linked by a peptide bond.

17. The linear therapeutic peptide of claim 16, further comprising a linker peptide positioned between the carrier peptide and the cargo peptide, wherein the carrier peptide and the cargo peptide are linked to the linker peptide by a peptide bond.

18. A therapeutic peptide multimer, comprising:
at least one carrier peptide conjugated to a plurality of PKC activity modulating cargo peptides.

19. The therapeutic peptide multimer of claim 18, wherein the at least one carrier protein comprises a first and a second cysteine residue.

20. The therapeutic peptide multimer of claim 19, wherein a first cargo peptide is linked to the first cysteine residue by a disulfide bond and a second cargo protein is linked to the second cysteine residue by a disulfide bond.


SEQUENCE LISTING (172 sequences)

SEQ ID NO: 1 - protein, 11 aa, synthetic construct
  REGION 1..11: note = Synthetic construct
  DOMAIN 1..11: note = Cargo peptide
CELGSLQAEDD

SEQ ID NO: 2 - protein, 8 aa, synthetic construct
  REGION 1..8: note = Synthetic construct
  DOMAIN 1..8: note = Cargo peptide
IPEGDEEG

SEQ ID NO: 3 - protein, 6 aa, synthetic construct
  REGION 1..6: note = Synthetic construct
  DOMAIN 1..6: note = Cargo peptide
QLVIAN

SEQ ID NO: 4 - protein, 6 aa, synthetic construct
  REGION 1..6: note = Synthetic construct
  DOMAIN 1..6: note = Cargo peptide
GLGAEN

SEQ ID NO: 5 - protein, 6 aa, synthetic construct
  REGION 1..6: note = Synthetic construct
  DOMAIN 1..6: note = Cargo peptide
ARGAEN

SEQ ID NO: 6 - protein, 7 aa, synthetic construct
  REGION 1..7: note = Synthetic construct
  DOMAIN 1..7: note = Cargo peptide
CGKGAEN

SEQ ID NO: 7 - protein, 7 aa, synthetic construct
  REGION 1..7: note = Synthetic construct
  DOMAIN 1..7: note = Cargo peptide
CGKGAEN

SEQ ID NO: 8 - protein, 8 aa, synthetic construct
  REGION 1..8: note = Synthetic construct
  DOMAIN 1..8: note = Cargo peptide
KQKTKTIK

SEQ ID NO: 9 - protein, 13 aa, synthetic construct
  REGION 1..13: note = Synthetic construct
  DOMAIN 1..13: note = Cargo peptide
MDPNGLSDPYVKL

SEQ ID NO: 10 - protein, 7 aa, synthetic construct
  REGION 1..7: note = Synthetic construct
  DOMAIN 1..7: note = Cargo peptide
IPDPKSE

SEQ ID NO: 11 - protein, 9 aa, synthetic construct
  REGION 1..9: note = Synthetic construct
  DOMAIN 1..9: note = Cargo peptide
SLNPEWNET

SEQ ID NO: 12 - protein, 8 aa, synthetic construct
  REGION 1..8: note = Synthetic construct
  DOMAIN 1..8: note = Cargo peptide
VPPEGSEA

SEQ ID NO: 13 - protein, 6 aa, synthetic construct
  REGION 1..6: note = Synthetic construct
  DOMAIN 1..6: note = Cargo peptide
KLFIMN

SEQ ID NO: 14 - protein, 7 aa, synthetic construct
  REGION 1..7: note = Synthetic construct
  DOMAIN 1..7: note = Cargo peptide
RDKRDTS

SEQ ID NO: 15 - protein, 9 aa, synthetic construct
  REGION 1..9: note = Synthetic construct
  DOMAIN 1..9: note = Cargo peptide
CARDKRDTS

SEQ ID NO: 16 - protein, 8 aa, synthetic construct
  REGION 1..8: note = Synthetic construct
  DOMAIN 1..8: note = Cargo peptide
GRDKRDTS

SEQ ID NO: 17 - protein, 8 aa, synthetic construct
  REGION 1..8: note = Synthetic construct
  DOMAIN 1..8: note = Cargo peptide
ARDKRDTS

SEQ ID NO: 18 - protein, 12 aa, synthetic construct
  REGION 1..12: note = Synthetic construct
  DOMAIN 1..12: note = Cargo peptide
ARDKRDTSNFDK

SEQ ID NO: 19 - protein, 14 aa, synthetic construct
  REGION 1..14: note = Synthetic construct
  DOMAIN 1..14: note = Cargo peptide
AGFSYTNPEFVINV

SEQ ID NO: 20 - protein, 6 aa, synthetic construct
  REGION 1..6: note = Synthetic construct
  DOMAIN 1..6: note = Cargo peptide
QEVIRN

SEQ ID NO: 21 - protein, 6 aa, synthetic construct
  REGION 1..6: note = Synthetic construct
  DOMAIN 1..6: note = Cargo peptide
CGRNAE

SEQ ID NO: 22 - protein, 7 aa, synthetic construct
  REGION 1..7: note = Synthetic construct
  DOMAIN 1..7: note = Cargo peptide
ACGRNAE

SEQ ID NO: 23 - protein, 7 aa, synthetic construct
  REGION 1..7: note = Synthetic construct
  DOMAIN 1..7: note = Cargo peptide
ACGKNAE

SEQ ID NO: 24 - protein, 8 aa, synthetic construct
  REGION 1..8: note = Synthetic construct
  DOMAIN 1..8: note = Cargo peptide
KACGRNAE

SEQ ID NO: 25 - protein, 7 aa, synthetic construct
  REGION 1..7: note = Synthetic construct
  DOMAIN 1..7: note = Cargo peptide
CGRNAEN

SEQ ID NO: 26 - protein, 7 aa, synthetic construct
  REGION 1..7: note = Synthetic construct
  DOMAIN 1..7: note = Cargo peptide
ACGRNAE

SEQ ID NO: 27 - protein, 14 aa, synthetic construct
  REGION 1..14: note = Synthetic construct
  DOMAIN 1..14: note = Cargo peptide
SFVNSEFLKPEVLS

SEQ ID NO: 28 - protein, 8 aa, synthetic construct
  REGION 1..8: note = Synthetic construct
  DOMAIN 1..8: note = Cargo peptide
VADADNCS

SEQ ID NO: 29 - protein, 6 aa, synthetic construct
  REGION 1..6: note = Synthetic construct
  DOMAIN 1..6: note = Cargo peptide
GRSGEN

SEQ ID NO: 30 - protein, 6 aa, synthetic construct
  REGION 1..6: note = Synthetic construct
  DOMAIN 1..6: note = Cargo peptide
GLSGEN

SEQ ID NO: 31 - protein, 6 aa, synthetic construct
  REGION 1..6: note = Synthetic construct
  DOMAIN 1..6: note = Cargo peptide
RLVLAS

SEQ ID NO: 32 - protein, 8 aa, synthetic construct
  REGION 1..8: note = Synthetic construct
  DOMAIN 1..8: note = Cargo peptide
PCGRSGEN

SEQ ID NO: 33 - protein, 11 aa, synthetic construct
  REGION 1..11: note = Synthetic construct
  DOMAIN 1..11: note = Cargo peptide
CSFNSYELGSL

SEQ ID NO: 34 - protein, 10 aa, synthetic construct
  REGION 1..10: note = Synthetic construct
  DOMAIN 1..10: note = Cargo peptide
SFNSYELGSL

SEQ ID NO: 35 - protein, 10 aa, synthetic construct
  REGION 1..10: note = Synthetic construct
  DOMAIN 1..10: note = Cargo peptide
TFNSYELGSL

SEQ ID NO: 36 - protein, 11 aa, synthetic construct
  REGION 1..11: note = Synthetic construct
  DOMAIN 1..11: note = Cargo peptide
AFNSNYELGSL

SEQ ID NO: 37 - protein, 10 aa, synthetic construct
  REGION 1..10: note = Synthetic construct
  DOMAIN 1..10: note = Cargo peptide
SFNSYELGTL

SEQ ID NO: 38 - protein, 10 aa, synthetic construct
  REGION 1..10: note = Synthetic construct
  DOMAIN 1..10: note = Cargo peptide
STNSYELGSL

SEQ ID NO: 39 - protein, 10 aa, synthetic construct
  REGION 1..10: note = Synthetic construct
  DOMAIN 1..10: note = Cargo peptide
SFNSFELGSL

SEQ ID NO: 40 - protein, 9 aa, synthetic construct
  REGION 1..9: note = Synthetic construct
  DOMAIN 1..9: note = Cargo peptide
SNSYDLGSL

SEQ ID NO: 41 - protein, 10 aa, synthetic construct
  REGION 1..10: note = Synthetic construct
  DOMAIN 1..10: note = Cargo peptide
SFNSYELPSL

SEQ ID NO: 42 - protein, 10 aa, synthetic construct
  REGION 1..10: note = Synthetic construct
  DOMAIN 1..10: note = Cargo peptide
TFNSYELGTL

SEQ ID NO: 43 - protein, 10 aa, synthetic construct
  REGION 1..10: note = Synthetic construct
  DOMAIN 1..10: note = Cargo peptide
SFNSYEIGSV

SEQ ID NO: 44 - protein, 10 aa, synthetic construct
  REGION 1..10: note = Synthetic construct
  DOMAIN 1..10: note = Cargo peptide
SFNSYEVGSI

SEQ ID NO: 45 - protein, 10 aa, synthetic construct
  REGION 1..10: note = Synthetic construct
  DOMAIN 1..10: note = Cargo peptide
SFNSYELGSV

SEQ ID NO: 46 - protein, 10 aa, synthetic construct
  REGION 1..10: note = Synthetic construct
  DOMAIN 1..10: note = Cargo peptide
SFNSYELGSI

SEQ ID NO: 47 - protein, 10 aa, synthetic construct
  REGION 1..10: note = Synthetic construct
  DOMAIN 1..10: note = Cargo peptide
SFNSYEIGSL

SEQ ID NO: 48 - protein, 10 aa, synthetic construct
  REGION 1..10: note = Synthetic construct
  DOMAIN 1..10: note = Cargo peptide
SFNSYEVGSL

SEQ ID NO: 49 - protein, 10 aa, synthetic construct
  REGION 1..10: note = Synthetic construct
  DOMAIN 1..10: note = Cargo peptide
AFNSYELGSL

SEQ ID NO: 50 - protein, 6 aa, synthetic construct
  REGION 1..6: note = Synthetic construct
  DOMAIN 1..6: note = Cargo peptide
YDLGSL

SEQ ID NO: 51 - protein, 6 aa, synthetic construct
  REGION 1..6: note = Synthetic construct
  DOMAIN 1..6: note = Cargo peptide
FDLGSL

SEQ ID NO: 52 - protein, 6 aa, synthetic construct
  REGION 1..6: note = Synthetic construct
  DOMAIN 1..6: note = Cargo peptide
YDIGSL

SEQ ID NO: 53 - protein, 6 aa, synthetic construct
  REGION 1..6: note = Synthetic construct
  DOMAIN 1..6: note = Cargo peptide
YDVGSL

SEQ ID NO: 54 - protein, 6 aa, synthetic construct
  REGION 1..6: note = Synthetic construct
  DOMAIN 1..6: note = Cargo peptide
YDLPSL

SEQ ID NO: 55 - protein, 6 aa, synthetic construct
  REGION 1..6: note = Synthetic construct
  DOMAIN 1..6: note = Cargo peptide
YDLGLL

SEQ ID NO: 56 - protein, 6 aa, synthetic construct
  REGION 1..6: note = Synthetic construct
  DOMAIN 1..6: note = Cargo peptide
YDLGSI

SEQ ID NO: 57 - protein, 6 aa, synthetic construct
  REGION 1..6: note = Synthetic construct
  DOMAIN 1..6: note = Cargo peptide
YDLGSV

SEQ ID NO: 58 - protein, 4 aa, synthetic construct
  REGION 1..4: note = Synthetic construct
  DOMAIN 1..4: note = Cargo peptide
IGSL

SEQ ID NO: 59 - protein, 4 aa, synthetic construct
  REGION 1..4: note = Synthetic construct
  DOMAIN 1..4: note = Cargo peptide
VGSL

SEQ ID NO: 60 - protein, 4 aa, synthetic construct
  REGION 1..4: note = Synthetic construct
  DOMAIN 1..4: note = Cargo peptide
LPSL

SEQ ID NO: 61 - protein, 4 aa, synthetic construct
  REGION 1..4: note = Synthetic construct
  DOMAIN 1..4: note = Cargo peptide
LGLL

SEQ ID NO: 62 - protein, 4 aa, synthetic construct
  REGION 1..4: note = Synthetic construct
  DOMAIN 1..4: note = Cargo peptide
LGSI

SEQ ID NO: 63 - protein, 4 aa, synthetic construct
  REGION 1..4: note = Synthetic construct
  DOMAIN 1..4: note = Cargo peptide
LGSV

SEQ ID NO: 64 - protein, 11 aa, synthetic construct
  REGION 1..11: note = Synthetic construct
  DOMAIN 1..11: note = Cargo peptide
ALSTERGKTLV

SEQ ID NO: 65 - protein, 11 aa, synthetic construct
  REGION 1..11: note = Synthetic construct
  DOMAIN 1..11: note = Cargo peptide
ALSTDRGKTLV

SEQ ID NO: 66 - protein, 11 aa, synthetic construct
  REGION 1..11: note = Synthetic construct
  DOMAIN 1..11: note = Cargo peptide
ALTSDRGKTLV

SEQ ID NO: 67 - protein, 11 aa, synthetic construct
  REGION 1..11: note = Synthetic construct
ALTTDRGKSLV

SEQ ID NO: 68 - protein, 11 aa, synthetic construct
  REGION 1..11: note = Synthetic construct
  DOMAIN 1..11: note = Cargo peptide
ALTTDRPKTLV

SEQ ID NO: 69 - protein, 11 aa, synthetic construct
  REGION 1..11: note = Synthetic construct
  DOMAIN 1..11: note = Cargo peptide
ALTTDRGRTLV

SEQ ID NO: 70 - protein, 11 aa, synthetic construct
  REGION 1..11: note = Synthetic construct
  DOMAIN 1..11: note = Cargo peptide
ALTTDKGKTLV

SEQ ID NO: 71 - protein, 11 aa, synthetic construct
  REGION 1..11: note = Synthetic construct
  DOMAIN 1..11: note = Cargo peptide
ALTTDKGKTLV

SEQ ID NO: 72 - protein, 10 aa, synthetic construct
  REGION 1..10: note = Synthetic construct
  DOMAIN 1..10: note = Cargo peptide
VLMRAAEEPV

SEQ ID NO: 73 - protein, 10 aa, synthetic construct
  REGION 1..10: note = Synthetic construct
  DOMAIN 1..10: note = Cargo peptide
QSMRSEDEAK

SEQ ID NO: 74 - protein, 9 aa, synthetic construct
  REGION 1..9: note = Synthetic construct
  DOMAIN 1..9: note = Cargo peptide
QGFEKKTGV

SEQ ID NO: 75 - protein, 9 aa, synthetic construct
  REGION 1..9: note = Synthetic construct
  DOMAIN 1..9: note = Cargo peptide
DNNGTYGKI

SEQ ID NO: 76 - protein, 6 aa, synthetic construct
  REGION 1..6: note = Synthetic construct
  DOMAIN 1..6: note = Cargo peptide
KNLIDS

SEQ ID NO: 77 - protein, 8 aa, synthetic construct
  REGION 1..8: note = Synthetic construct
  DOMAIN 1..8: note = Cargo peptide
VKSPRDYS

SEQ ID NO: 78 - protein, 9 aa, synthetic construct
  REGION 1..9: note = Synthetic construct
  DOMAIN 1..9: note = Cargo peptide
VKSPCRDYS

SEQ ID NO: 79 - protein, 8 aa, synthetic construct
  REGION 1..8: note = Synthetic construct
  DOMAIN 1..8: note = Cargo peptide
IKSPRLYS

SEQ ID NO: 80 - protein, 6 aa, synthetic construct
  REGION 1..6: note = Synthetic construct
  DOMAIN 1..6: note = Cargo peptide
KNLIDS

SEQ ID NO: 81 - protein, 11 aa, synthetic construct
  REGION 1..11: note = Synthetic construct
  DOMAIN 1..11: note = Cargo peptide
PKVKSPRDYSN

SEQ ID NO: 82 - protein, 7 aa, synthetic construct
  REGION 1..7: note = Synthetic construct
  DOMAIN 1..7: note = Cargo peptide
NGLLKIK

SEQ ID NO: 83 - protein, 8 aa, synthetic construct
  REGION 1..8: note = Synthetic construct
  DOMAIN 1..8: note = Cargo peptide
EAVSLKPT

SEQ ID NO: 84 - protein, 11 aa, synthetic construct
  REGION 1..11: note = Synthetic construct
  DOMAIN 1..11: note = Cargo peptide
LAVFHDAPIGY

SEQ ID NO: 85 - protein, 9 aa, synthetic construct
  REGION 1..9: note = Synthetic construct
  DOMAIN 1..9: note = Cargo peptide
DDFVANCTI

SEQ ID NO: 86 - protein, 10 aa, synthetic construct
  REGION 1..10: note = Synthetic construct
  DOMAIN 1..10: note = Cargo peptide
WIDLEPEGRV

SEQ ID NO: 87 - protein, 10 aa, synthetic construct
  REGION 1..10: note = Synthetic construct
  DOMAIN 1..10: note = Cargo peptide
HAVGPRPQTF

SEQ ID NO: 88 - protein, 8 aa, synthetic construct
  REGION 1..8: note = Synthetic construct
  DOMAIN 1..8: note = Cargo peptide
NGSRHFED

SEQ ID NO: 89 - protein, 8 aa, synthetic construct
  REGION 1..8: note = Synthetic construct
  DOMAIN 1..8: note = Cargo peptide
HDAPIGYD

SEQ ID NO: 90 - protein, 6 aa, synthetic construct
  REGION 1..6: note = Synthetic construct
  DOMAIN 1..6: note = Cargo peptide
HDAPIG

SEQ ID NO: 91 - protein, 8 aa, synthetic construct
  REGION 1..8: note = Synthetic construct
  DOMAIN 1..8: note = Cargo peptide
HDAAIGYD

SEQ ID NO: 92 - protein, 8 aa, synthetic construct
  REGION 1..8: note = Synthetic construct
  DOMAIN 1..8: note = Cargo peptide
HDAPIPYD

SEQ ID NO: 93 - protein, 8 aa, synthetic construct
  REGION 1..8: note = Synthetic construct
  DOMAIN 1..8: note = Cargo peptide
HNAPIGYD

SEQ ID NO: 94 - protein, 8 aa, synthetic construct
  REGION 1..8: note = Synthetic construct
  DOMAIN 1..8: note = Cargo peptide
HAAPIGYD

SEQ ID NO: 95 - protein, 8 aa, synthetic construct
  REGION 1..8: note = Synthetic construct
  DOMAIN 1..8: note = Cargo peptide
ADAPIGYD

SEQ ID NO: 96 - protein, 8 aa, synthetic construct
  REGION 1..8: note = Synthetic construct
  DOMAIN 1..8: note = Cargo peptide
HDAPAGYD

SEQ ID NO: 97 - protein, 8 aa, synthetic construct
  REGION 1..8: note = Synthetic construct
  DOMAIN 1..8: note = Cargo peptide
HDAPIGAD

SEQ ID NO: 98 - protein, 8 aa, synthetic construct
  REGION 1..8: note = Synthetic construct
  DOMAIN 1..8: note = Cargo peptide
HDAPIAYD

SEQ ID NO: 99 - protein, 8 aa, synthetic construct
  REGION 1..8: note = Synthetic construct
  DOMAIN 1..8: note = Cargo peptide
HDAPIGYA

SEQ ID NO: 100 - protein, 9 aa, synthetic construct
  REGION 1..9: note = Synthetic construct
  DOMAIN 1..9: note = Cargo peptide
SSPSEEDRS

SEQ ID NO: 101 - protein, 8 aa, synthetic construct
  REGION 1..8: note = Synthetic construct
  DOMAIN 1..8: note = Cargo peptide
PCDQEIKE

SEQ ID NO: 102 - protein, 9 aa, synthetic construct
  REGION 1..9: note = Synthetic construct
  DOMAIN 1..9: note = Cargo peptide
ENNIRKALS

SEQ ID NO: 103 - protein, 8 aa, synthetic construct
  REGION 1..8: note = Synthetic construct
  DOMAIN 1..8: note = Cargo peptide
GEVRQGQA

SEQ ID NO: 104 - protein, 6 aa, synthetic construct
  REGION 1..6: note = Synthetic construct
  DOMAIN 1..6: note = Cargo peptide
EAIVKQ

SEQ ID NO: 105 - protein, 7 aa, synthetic construct
  REGION 1..7: note = Synthetic construct
  DOMAIN 1..7: note = Cargo peptide
IKTKRDV

SEQ ID NO: 106 - protein, 7 aa, synthetic construct
  REGION 1..7: note = Synthetic construct
  DOMAIN 1..7: note = Cargo peptide
IKTKRLI

SEQ ID NO: 107 - protein, 7 aa, synthetic construct
  REGION 1..7: note = Synthetic construct
  DOMAIN 1..7: note = Cargo peptide
CEAIVKQ

SEQ ID NO: 108 - protein, 10 aa, synthetic construct
  REGION 1..10: note = Synthetic construct
  DOMAIN 1..10: note = Cargo peptide
TKRDVNNFDQ

SEQ ID NO: 109 - protein, 7 aa, synthetic construct
  REGION 1..7: note = Synthetic construct
  DOMAIN 1..7: note = Cargo peptide
VRLKAHY

SEQ ID NO: 110 - protein, 6 aa, synthetic construct
  REGION 1..6: note = Synthetic construct
  DOMAIN 1..6: note = Cargo peptide
VDSEGD

SEQ ID NO: 111 - protein, 8 aa, synthetic construct
  REGION 1..8: note = Synthetic construct
  DOMAIN 1..8: note = Cargo peptide
VFPSIPEQ

SEQ ID NO: 112 - protein, 15 aa, synthetic construct
  REGION 1..15: note = Synthetic construct
  DOMAIN 1..15: note = Cargo peptide
SQEPPVDDKNEDADL

SEQ ID NO: 113 - protein, 7 aa, synthetic construct
  REGION 1..7: note = Synthetic construct
  DOMAIN 1..7: note = Cargo peptide
IKDDSED

SEQ ID NO: 114 - protein, 9 aa, synthetic construct
  REGION 1..9: note = Synthetic construct
  DOMAIN 1..9: note = Cargo peptide
PVIDGMDGI

SEQ ID NO: 115 - protein, 6 aa, synthetic construct
  REGION 1..6: note = Synthetic construct
  DOMAIN 1..6: note = Cargo peptide
EDAIKR

SEQ ID NO: 116 - protein, 5 aa, synthetic construct
  REGION 1..5: note = Synthetic construct
  DOMAIN 1..5: note = Cargo peptide
EDAIR

SEQ ID NO: 117 - protein, 8 aa, synthetic construct
  REGION 1..8: note = Synthetic construct
  DOMAIN 1..8: note = Cargo peptide
ITDDYGLD

SEQ ID NO: 118 - protein, 8 aa, synthetic construct
  REGION 1..8: note = Synthetic construct
  DOMAIN 1..8: note = Cargo peptide
ITDDYGDL

SEQ ID NO: 119 - protein, 7 aa, synthetic construct
  REGION 1..7: note = Synthetic construct
  DOMAIN 1..7: note = Cargo peptide
DDYGLDN

SEQ ID NO: 120 - protein, 7 aa, synthetic construct
  REGION 1..7: note = Synthetic construct
  DOMAIN 1..7: note = Cargo peptide
NGYLRVR

SEQ ID NO: 121 - protein, 8 aa, synthetic construct
  REGION 1..8: note = Synthetic construct
  DOMAIN 1..8: note = Cargo peptide
EAVGLQPT

SEQ ID NO: 122 - protein, 11 aa, synthetic construct
  REGION 1..11: note = Synthetic construct
  DOMAIN 1..11: note = Cargo peptide
LAVFHETPLGY

SEQ ID NO: 123 - protein, 8 aa, synthetic construct
  REGION 1..8: note = Synthetic construct
  DOMAIN 1..8: note = Cargo peptide
DFVANCTL

SEQ ID NO: 124 - protein, 10 aa, synthetic construct
  REGION 1..10: note = Synthetic construct
  DOMAIN 1..10: note = Cargo peptide
WVDLEPEGKV

SEQ ID NO: 125 - protein, 8 aa, synthetic construct
  REGION 1..8: note = Synthetic construct
  DOMAIN 1..8: note = Cargo peptide
HSLFKKGH

SEQ ID NO: 126 - protein, 9 aa, synthetic construct
  REGION 1..9: note = Synthetic construct
  DOMAIN 1..9: note = Cargo peptide
TGASDTFEG

SEQ ID NO: 127 - protein, 6 aa, synthetic construct
  REGION 1..6: note = Synthetic construct
  DOMAIN 1..6: note = Cargo peptide
EGHLPM

SEQ ID NO: 128 - protein, 6 aa, synthetic construct
  REGION 1..6: note = Synthetic construct
  DOMAIN 1..6: note = Cargo peptide
EGHDPM

SEQ ID NO: 129 - protein, 8 aa, synthetic construct
  REGION 1..8: note = Synthetic construct
  DOMAIN 1..8: note = Cargo peptide
IKSREDVS

SEQ ID NO: 130 - protein, 8 aa, synthetic construct
  REGION 1..8: note = Synthetic construct
  DOMAIN 1..8: note = Cargo peptide
VRSREDVS

SEQ ID NO: 131 - protein, 9 aa, synthetic construct
  REGION 1..9: note = Synthetic construct
  DOMAIN 1..9: note = Cargo peptide
PRIKSREDV

SEQ ID NO: 132 - protein, 10 aa, synthetic construct
  REGION 1..10: note = Synthetic construct
  DOMAIN 1..10: note = Cargo peptide
HQVRVKAYYR

SEQ ID NO: 133 - protein, 11 aa, synthetic construct
  REGION 1..11: note = Synthetic construct
  DOMAIN 1..11: note = Cargo peptide
YELNKDSELLI

SEQ ID NO: 134 - protein, 15 aa, synthetic construct
  REGION 1..15: note = Synthetic construct
  DOMAIN 1..15: note = Cargo peptide
MDQSSMHSDHAQTVI

SEQ ID NO: 135 - protein, 7 aa, synthetic construct
  REGION 1..7: note = Synthetic construct
  DOMAIN 1..7: note = Cargo peptide
LDQVGEE

SEQ ID NO: 136 - protein, 9 aa, synthetic construct
  REGION 1..9: note = Synthetic construct
  DOMAIN 1..9: note = Cargo peptide
EAMNTRESG

SEQ ID NO: 137 - protein, 6 aa, synthetic construct
  REGION 1..6: note = Synthetic construct
  DOMAIN 1..6: note = Cargo peptide
DDIVRK

SEQ ID NO: 138 - protein, 8 aa, synthetic construct
  REGION 1..8: note = Synthetic construct
  DOMAIN 1..8: note = Cargo peptide
VKLCDFGF

SEQ ID NO: 139 - protein, 8 aa, synthetic construct
  REGION 1..8: note = Synthetic construct
  DOMAIN 1..8: note = Cargo peptide
IRLCDFAF

SEQ ID NO: 140 - protein, 10 aa, synthetic construct
  REGION 1..10: note = Synthetic construct
  DOMAIN 1..10: note = Cargo peptide
QVKLCDFGFA

SEQ ID NO: 141 - protein, 9 aa, synthetic construct
  REGION 1..9: note = Synthetic construct
  DOMAIN 1..9: note = Cargo peptide
MSVPPLLRP

SEQ ID NO: 142 - protein, 11 aa, synthetic construct
  REGION 1..11: note = Synthetic construct
  DOMAIN 1..11: note = Cargo peptide
KFPECGFYGLY

SEQ ID NO: 143 - protein, 9 aa, synthetic construct
  REGION 1..9: note = Synthetic construct
  DOMAIN 1..9: note = Cargo peptide
DPDADQEDS

SEQ ID NO: 144 - protein, 9 aa, synthetic construct
  REGION 1..9: note = Synthetic construct
  DOMAIN 1..9: note = Cargo peptide
SKDTLRKRH

SEQ ID NO: 145 - protein, 9 aa, synthetic construct
  REGION 1..9: note = Synthetic construct
  DOMAIN 1..9: note = Cargo peptide
ITLFQNDTG

SEQ ID NO: 146 - protein, 9 aa, synthetic construct
  REGION 1..9: note = Synthetic construct
  DOMAIN 1..9: note = Cargo peptide
GSNSHKDIS

SEQ ID NO: 147 - protein, 6 aa, synthetic construct
  REGION 1..6: note = Synthetic construct
  DOMAIN 1..6: note = Cargo peptide
SDSPEA

SEQ ID NO: 148 - protein, 8 aa, synthetic construct
  REGION 1..8: note = Synthetic construct
  DOMAIN 1..8: note = Cargo peptide
GLSNFDCG

SEQ ID NO: 149 - protein, 11 aa, synthetic construct
  REGION 1..11: note = Synthetic construct
  DOMAIN 1..11: note = Cargo peptide
YVESENGQMYI

SEQ ID NO: 150 - protein, 10 aa, synthetic construct
  REGION 1..10: note = Synthetic construct
  DOMAIN 1..10: note = Cargo peptide
IVKGKNVDLI

SEQ ID NO: 151 - protein, 10 aa, synthetic construct
  REGION 1..10: note = Synthetic construct
  DOMAIN 1..10: note = Cargo peptide
DMNEFETEGF

SEQ ID NO: 152 - protein, 9 aa, synthetic construct
  REGION 1..9: note = Synthetic construct
  DOMAIN 1..9: note = Cargo peptide
CSIKNEARL

SEQ ID NO: 153 - protein, 9 aa, synthetic construct
  REGION 1..9: note = Synthetic construct
  DOMAIN 1..9: note = Cargo peptide
GKREPQGIS

SEQ ID NO: 154 - protein, 9 aa, synthetic construct
  REGION 1..9: note = Synthetic construct
  DOMAIN 1..9: note = Cargo peptide
DEVDKMCHL

SEQ ID NO: 155 - protein, 6 aa, synthetic construct
  REGION 1..6: note = Synthetic construct
  DOMAIN 1..6: note = Cargo peptide
RALINS

SEQ ID NO: 156 - protein, 8 aa, synthetic construct
  REGION 1..8: note = Synthetic construct
  DOMAIN 1..8: note = Cargo peptide
VKSPFDCS

SEQ ID NO: 157 - protein, 8 aa, synthetic construct
  REGION 1..8: note = Synthetic construct
  DOMAIN 1..8: note = Cargo peptide
VRSPFDCS

SEQ ID NO: 158 - protein, 7 aa, synthetic construct
  REGION 1..7: note = Synthetic construct
  DOMAIN 1..7: note = Cargo peptide
DRALINS

SEQ ID NO: 159 - protein, 8 aa, synthetic construct
  REGION 1..8: note = Synthetic construct
  DOMAIN 1..8: note = Cargo peptide
ISGEFGLD

SEQ ID NO: 160 - protein, 8 aa, synthetic construct
  REGION 1..8: note = Synthetic construct
  DOMAIN 1..8: note = Cargo peptide
CSGEFGLD

SEQ ID NO: 161 - protein, 7 aa, synthetic construct
  REGION 1..7: note = Synthetic construct
  DOMAIN 1..7: note = Cargo peptide
DDDIVRK

SEQ ID NO: 162 - protein, 6 aa, synthetic construct
  REGION 1..6: note = Synthetic construct
  DOMAIN 1..6: note = Cargo peptide
DDIVRK

SEQ ID NO: 163 - protein, 9 aa, synthetic construct
  REGION 1..9: note = Synthetic construct
  DOMAIN 1..9: note = Cargo peptide
AFNSYELGS

SEQ ID NO: 164 - protein, 12 aa, synthetic construct
  REGION 1..12: note = Synthetic construct
  DOMAIN 1..12: note = Carrier peptide
CYGRKKRRQRRR

SEQ ID NO: 165 - protein, 10 aa, synthetic construct
  REGION 1..10: note = Synthetic construct
  DOMAIN 1..10: note = Cargo peptide
CSFNSYELGS

SEQ ID NO: 166 - protein, 11 aa, synthetic construct
  REGION 1..11: note = Synthetic construct
  DOMAIN 1..11: note = Carrier peptide
YGRKKRRQRRR

SEQ ID NO: 167 - protein, 21 aa, synthetic construct
  REGION 1..21: note = Synthetic construct
  DOMAIN 1..21: note = Linear peptide
EAVSLKPTGGYGRKKRRQRRR

SEQ ID NO: 168 - protein, 7 aa, synthetic construct
  REGION 1..7: note = Synthetic construct
  DOMAIN 1..7: note = Carrier peptide
CKLFIMN

SEQ ID NO: 169 - protein, 20 aa, synthetic construct
  REGION 1..20: note = Synthetic construct
  DOMAIN 1..20: note = Cargo peptide
GKLFIMNLGYGRKKRRQRRR

SEQ ID NO: 170 - protein, 21 aa, synthetic construct
  REGION 1..21: note = Synthetic construct
  DOMAIN 1..21: note = Linear peptide
GKLFIMNLSGYGRKKRRQRRR

SEQ ID NO: 171 - protein, 21 aa, synthetic construct
  REGION 1..21: note = Synthetic construct
  DOMAIN 1..21: note = Linear peptide
GEEVIRNISGYGRKKRRQRRR

SEQ ID NO: 172 - protein, 12 aa, synthetic construct
  REGION 1..12: note = Synthetic construct
  DOMAIN 1..12: note = Modified TAT carrier peptide
  SITE 1: note = Xaa = acetylated cysteine
  SITE 12: note = Xaa = amide-modified arginine
XYGRKKRRQRRX